SEQUENCE LISTING (18 sequences)

SEQ ID NO: 1 - DNA, 33 bp, Artificial Sequence
  Description of Artificial Sequence left element (LE) loxP site (also known as lox71)
taccgttcgtatagcatcattatacgaagttat

SEQ ID NO: 2 - DNA, 33 bp, Artificial Sequence
  Description of Artificial Sequence right element (RE) loxP site (also known as lox66)
ataacttcgtatagcatcattatacgaacggta

SEQ ID NO: 3 - DNA, 27 bp, Artificial Sequence
  Description of Artificial Sequence Primer
catggtcaggtcatggatgagcagacg

SEQ ID NO: 4 - DNA, 28 bp, Artificial Sequence
  Description of Artificial Sequence Primer
ctgccgggatactcgtttaatgcccatc

SEQ ID NO: 5 - DNA, 26 bp, Artificial Sequence
  Description of Artificial Sequence Primer
gcgccaccatccagtgcaggagctcg

SEQ ID NO: 6 - DNA, 100 bp, Artificial Sequence
  Description of Artificial Sequence Oligonucleotide
ggaattcataacttcgtataatgtatgctatacgaagttattaatctcgagtaataacttcgtataatgtatgctatacgaagttatggtaccgcgcccg

SEQ ID NO: 7 - DNA, 19 bp, Artificial Sequence
  Description of Artificial Sequence Oligonucleotide
cgggcgcggtaccataact

SEQ ID NO: 8 - DNA, 47 bp, Artificial Sequence
  Description of Artificial Sequence Oligonucleotide
tcgagtcgactctagaggatccgagctccccgggaagcttctgcagt

SEQ ID NO: 9 - DNA, 47 bp, Artificial Sequence
  Description of Artificial Sequence Oligonucleotide
tcgaactgcagaagcttcccggggagctcggatcctctagagtcgac

SEQ ID NO: 10 - DNA, 94 bp, Artificial Sequence
  Description of Artificial Sequence Oligonucleotide
ctcgaattcataacttcgtatagcatacattatacgaacggtactcgagtaccgttcgtatagcatacattatacgaagttatggtaccaaaaa

SEQ ID NO: 11 - DNA, 17 bp, Artificial Sequence
  Description of Artificial Sequence Oligonucleotide
tttttggtaccataact

SEQ ID NO: 12 - DNA, 21 bp, Artificial Sequence
  Description of Artificial Sequence Primer
gtgaatgatgtagccgtcaag

SEQ ID NO: 13 - DNA, 18 bp, Artificial Sequence
  Description of Artificial Sequence Primer
ccatgattacggattcac

SEQ ID NO: 14 - DNA, 35 bp, Artificial Sequence
  Description of Artificial Sequence Primer
agcgatggtaccatgagtcacccagatctaaataa

SEQ ID NO: 15 - DNA, 35 bp, Artificial Sequence
  Description of Artificial Sequence Primer
taggttggtaccttagtaaatctttgtcccatgtt

SEQ ID NO: 16 - DNA, 94 bp, Artificial Sequence; identical to SEQ ID NO: 10
  Description of Artificial Sequence Oligonucleotide

SEQ ID NO: 17 - DNA, 18 bp, Artificial Sequence
  Description of Artificial Sequence Oligonucleotide
tgatgaattccgcgcccg

SEQ ID NO: 18 - DNA, 34 bp, Artificial Sequence
  Description of Artificial Sequence Construct
ataacttcgtatagcatacattatacgaacggta

(19) United States
(12) Patent Application Publication
(10) Pub. No.: US 2017/0342424 A1
(43) Pub. Date: Nov. 30, 2017

(54) METHODS OF PRODUCING RECOMBINANT MINICIRCLE CONSTRUCTS

(71) Applicant: PLASMIDFACTORY GMBH CO & KG, BIELEFELD (DE)

(72) Inventors: Brian W. Bigger, London (GB); Oleg Tolmachov, London (GB); Charles Coutelle, London (GB)

(73) Assignee: PLASMIDFACTORY GMBH CO & KG, BIELEFELD (DE)

(21) Appl. No.: 15/157,756

(22) Filed: May 18, 2016

Related U.S. Application Data
(63) Continuation of application No. 11/249,929, filed on Oct. 13, 2005, now abandoned.
Application No. 11/249,929 is a continuation of application No. 10/118,231, filed on Apr. 9, 2002, now abandoned.
Application No. 11/249,929 is a continuation of application No. 10/118,231, filed on Apr. 9, 2002, now abandoned.
(60) Provisional application No. 60/327,029, filed on Oct. 5, 2001.
(60) Provisional application No. 60/327,029, filed on Oct. 5, 2001.

(30) Foreign Application Priority Data
Apr. 10, 2001 (GB) 0108968.9

(51) Int. Cl. C12N 15/79

(52) U.S. Cl. CPC C12N 15/79

(57) ABSTRACT

The present invention relates to a method for the production of a minicircle. In the method of the present invention, a parent plasmid is provided which has a nucleic sequence flanked by recombination sites. This parent plasmid is exposed to an enzyme which causes recombination at the recombination sites, thereby to form a (i) minicircle comprising the nucleic acid sequence and (ii) a miniplasmid comprising the remainder of the parent plasmid. One recombination site is modified at the 5′ end such that its reaction with the enzyme is less efficient than the wild type site, and the other recombination site is modified at the 3′ end such that its reaction with the enzyme is less efficient than the wild type site, and the other recombination site is modified at the 3′ end such that its reaction with the enzyme is less efficient than the wild type site, both modified sites being located in the minicircle after recombination. This favours the formation of minicircle.

The present application is a divisional of U.S. application Ser. No. 11/249,929, filed Oct. 13, 2005, which is a continuation of U.S. application Ser. No. 10/118,231, filed Apr. 8, 2002, both of which claim the benefit of UK patent application number 0108968.9, filed Apr. 10, 2001, and U.S. provisional patent application Ser. No. 60/327,029, filed Oct. 5, 2001, all of which are hereby incorporated by reference in their entirety.

The present invention relates to recombinant minicircle constructs.

There is increasing evidence to suggest that plasmid DNA used for non-viral gene delivery can cause unacceptable inflammatory responses in eukaryotes (Krieg, (1996) J Lab Clin Med 128(2), 128-33; Yew, et al. (1999) Hum Gene Ther 10(2), 223-34; Norman, et al. (2000) Gene Ther 7(16), 1425-30; McLachlan, et al. (2000) Gene Ther 7(5), 384-92; Krieg, (1999) J Gene Med 1(1), 56-63). These immunotoxic responses are largely due to the presence of unmethylated CpG motifs and their associated stimulatory sequences on plasmids following bacterial propagation of plasmid DNA. Simple methylation of DNA in vitro may be enough to reduce an inflammatory response but is likely to result in severely depressed gene expression (Krieg, (2000) Mol Ther 1(3), 209-10). The removal of CpG islands by cloning out, or elimination of non-essential sequences is more successful in reducing inflammatory responses but is time-consuming and tedious (Yew, et al. (2000) Mol Ther 1(3), 255-62).

Since bacterial DNA contains on average 4 times more CpG islands than mammalian DNA (Swartz & Kornberg, (1962) J Biol Chem 237, 1961-1967), a good solution is to eliminate entirely the bacterial control regions, such as the origin of replication and antibiotic resistance genes, from gene delivery vectors during the process of plasmid production. Thus, the “parent” plasmid is recombined into a “minicircle” which generally comprises the gene to be delivered and suitable control regions for its expression, and a miniplasmid which generally comprises the remainder of the parent plasmid.

Removal of bacterial sequences needs to be efficient, using the smallest possible excision site, whilst creating supercoiled DNA minicircles which consist solely of gene expression elements under appropriate—preferably mammalian—control regions.

Previous techniques for minicircle production (Darquet, et al. (1997) Gene Ther 4(12), 1341-9; Darquet, et al. (1999) Gene Ther 6(2), 209-18; Kreiss, et al. (1998) Appl Micbiol Biotechnol 49(5), 560-7), have used bacterial phage λ integrase mediated recombination to produce minicircle DNA. This system results in attL or attR excision sites of 100-165 bp following recombination (Landy, (1989) Annu Rev Biochem 58, 913-49).

Cre recombinase is a bacteriophage P1 derived integrase (Abremski, et al. (1983) Cell 32(4), 1301-11; Abremski & Hoess, (1984) J Biol Chem 259(3), 1509-14; Sternberg, et al. (1986) J Mol Bio 187(2), 197-212) which catalyses site-specific recombination between direct repeats of 34 base pairs (loxP sites). The use of the Cre/lox system for the production of minicircles has been suggested (Bigger et al, 8^{the} Meeting of the ESGT, John Wiley & sons, Ltd, 2000). In the case of a supercoiled plasmid containing DNA flanked by two loxP sites in the same orientation, Cre recombination produces two DNA molecules that are topologically unlinked, circular, and mainly supercoiled (Abremski & Hoess, (1984) J Biol Chem 259(3), 1509-14), each containing a single 34 bp loxP site. When used in the production of a minicircle, it results in a recognition site of only 34 bp (Abremski, et al. (1983) Cell 32(4), 1301-11; Abremski & Hoess, (1984) J Biol Chem 259(3), 1509-14; Sternberg, et al. (1986) J Mol Biol 187(2), 197-212), thus producing a minimal construct size.

Cre recombinase normally leads to an equilibrium reaction of recombination mediating excision as well as insertion. It is therefore desirable to drive the equilibrium towards the production of the minicircle so as to improve yield.

### SUMMARY OF THE INVENTION

In a first aspect of the present invention, there is provided a method for the production of a minicircle, which method comprises: (a) providing a parent plasmid which has a nucleic sequence flanked by recombination sites; and (b) exposing the parent plasmid to an enzyme which causes recombination at the recombination sites, thereby to form (i) a minicircle comprising the nucleic acid sequence and (ii) a miniplasmid comprising the remainder of the parent plasmid, wherein one recombination site is modified at the 5′ end such that its reaction with the enzyme is less efficient than the wild type site, and the other recombination site is modified at the 3′ end such that its reaction with the enzyme is less efficient than the wild type site, both modified sites being located in the minicircle after recombination.

In a second aspect, the invention provides a nucleic acid construct comprising a nucleic acid sequence of interest flanked by two recombination sites, one recombination site being modified at the 5′ end such that its reaction with an enzyme which causes recombination at the recombination site is less efficient than the wild type site, and the other recombination site being modified at the 3′ end such that its reaction with the enzyme is less efficient than the wild type site. The invention also provides a cell, such as a bacterium, comprising such a construct.

Because the minicircle resulting from the recombination has both of the modified sites which react less efficiently with the enzyme, the reaction equilibrium is shifted towards increased production of minicircle.

In a preferred embodiment, the enzyme is Cre recombinase and the recombination sites are loxP sites. Thus, the parent plasmid contains two loxP sites, between which the nucleic acid (preferably DNA) that is to be recombined out is located. Cre-recombination leads to two nucleic acid circles (the minicircle and the miniplasmid), each containing one loxP site at which they can recombine. In this embodiment of the invention, the 3′ or the 5′ sequence of the two loxP sites in the production plasmid are mutated to have a less efficient reaction with Cre than wild-type loxP sites. With only one end mutated, the loxP sites both still function well as recognition sites for Cre. However, after recombination, both mutated ends form the loxP site in the minicircle, thereby reducing its ability to recombine with the fully normal loxP sites in the miniplasmid which are generated from the normal unmodified ends of the two recognition sites. In this way, the equilibrium is shifted towards generation of the minicircle.

The loxP sites may be modified as described in Albert, et al. (1995) Plant J 7(4), 649-59 and Araki, et al. (1997) Nucleic Acids Res 25(4), 868-72. In this regard, the terminal 5 nucleotides on one side of the loxP site may be modified to create a left element (LE) loxP site (also known as lox71), having the sequence TACCGTTCGTATA GCATCAT TATACGAAGTTAT, wherein the nucleic acids in bold are modified. The terminal 5 nucleotides on the other side of the loxP site may be modified to create a right element (RE) loxP site (also known as lox66), having the sequence ATAACTTCGTATA GCATCAT TATACGAACGGTA, wherein the nucleic acids in bold are modified.

The yield of minicircle DNA can be increased and degree of concatemerisation of minicircle DNA can be modified by varying the genotype of the minicircle-producing bacterial strain. For example, we have recently constructed a recA-minicircle producer bacterium based on E. coliK12 HB101. In this bacterium, the minicircle yield is increased because of reduced homologous recombination between minicircle and parental plasmid. Minicircle DNA produced in our recA-producer strain has reduced level of concatemerisation. If a high level of concatemerisation is desired, which may be beneficial for transcription in mammalian cells, the genotypes of the minicircle producing organism and parental plasmid can be varied to allow recombination dependent plasmid replication (Viret et al Microbiol Rev 1991 55(4):675-83).

The method is preferably carried out in a bacterium. E. coli is preferred. Streptomyces or glutamic acid-producing bacteria may also be used, although these are less preferred because their thick cell wall means that extraction of DNA is more difficult, leading to lower yield and poorer quality.

When the method is carried out in a bacterium, it is preferred if the bacterium expresses the Cre recombinase. The bacterium may be transformed to express the gene encoding the Cre recombinase. The gene may be inserted into the bacterial genome or may be expressed from a plasmid.

In order to avoid to premature Cre-recombination, resulting in loss of the replication-deficient minicircle due to out-competition by the replication-competent and antibiotic-resistant bacterial vector, it is preferred if expression of the Cre recombinase gene is controlled. Such controlled expression may be achieved by placing the cre recombinase gene under the control of a constitutive or inducible promoter. A preferred system useful for such tight transcriptional control is the arabinose expression system (Buchholz, et al. (1996) Nucleic Acids Res 24(21), 4256-62; Hirsh & Schleif, (1973) J Mol Biol 80(3), 433-44; Hahn, et al. (1984) J Mol Biol 180(1), 61-72; Kosiba & Schleif, (1982) J Mol Biol 156(1), 53-66, for review see Neidhardt, F. C. (ed) (1987) Esherichia coli and Salmonella typhimurium, cellular and molecular biology Vol. 2. Edited by Ingraham, et al. 2 vols., American Society for Microbiology, Washington D.C.), as this provides a cre expressing bacterial strain which is both stable and easily controllable by altering the carbon source available for metabolism by these bacteria. Alternative suitable transcriptional control systems include the operator-repressor system of phage λ (Breitling et al. 1990. Gene. 93(1) 35-40), the operator-repressor system of lac operon (Gronenborn 1976. Mol Gen Genet. 148. No. 3: 243-50; Yansura & Henner 1984 Proc Natl Acad Sci U.S.A. 81 (2):439-43), and the tetracycline repressor-operator system (Skerra 1994. Gene 151(1-2): 131-135. Other suitable transcriptional control systems are described in Gossen et al. Trends Biochem Sci 1993; 18 (12): 471-5; Gossen & Bujard Proc Natl Acad Sci USA 1992; 89 (12): 5547-51; Fussenegger et al. Nature Biotechnology. 2000. 18: 1203-1208; Plasterk et al Proc. Natl. Acad. Sci. USA. 1984. 81(9): 2689-2692; Kanegae et al Nucleic Acids Research. 1995. 23(19): 3816-3821; Kano et al Biochem. Biophys. Research Communications. 1998. 248: 806-811.

The method of the first aspect of the invention may further comprise exposing the minicircle and miniplasmid to one or more nucleic acid endonuclease(s), the parent plasmid having recognition site(s) of the endonuclease(s) located outside of the recombination sites and nucleic acid sequence. Alternatively, an exonuclease may be used: in the following, reference is made to an endonuclease for convenience. However, it is to be understood that an exonuclease can be used in place of the endonuclease. In this way, the endonuclease(s) (which are preferably DNA endonucleases) can specifically destroy the bacterial sequences in the miniplasmid and any remaining parent plasmid left over after production of the minicircle. This makes isolation of the resulting minicircle much simpler because it allows separation from the parent plasmid and the miniplasmid (the minicircle being the only non-linear nucleic acid).

In a preferred embodiment, the bacterium which produces the minicircles is engineered to express the endonuclease(s). The bacterium may be transformed or transfected to express the gene(s) encoding the endonuclease(s). The gene(s) may be inserted into the bacterial genome or may be expressed from a nucleic acid construct.

According to a fourth aspect of the invention, there is provided a cell, such as a bacterium, which (a) includes a parent plasmid which is capable of being specifically recombined to form a minicircle and a miniplasmid, and (b) is capable of expressing at least one endonuclease, wherein the parent plasmid and the miniplasmid have recognition site(s) of the endonuclease, and the minicircle does not have recognition site(s) of the endonuclease.

According to a fifth aspect of the present invention, there is provided a method for the production of a minicircle, which method comprises: (a) providing a cell of the fourth aspect of the invention; (b) causing the parent plasmid to be recombined to form (i) a minicircle comprising the nucleic acid sequence and (ii) a miniplasmid comprising the remainder of the parent plasmid; and (c) causing the bacterium to express at least one endonuclease.

It is preferred if, although not essential that, the or each gene for the endonuclease is placed under tight transcriptional control so that the endonuclease can be activated only after the minicircle has been produced from the minicircle producing plasmid, i.e. to avoid destruction of the minicircle producing plasmid before production of the minicircle. For example, operator-repressor system of phage lambda, operator-repressor system of lac operon, operator-activator system of araBAD operon, tetracycline repressor-operator system can be used for this purpose. Close control of endonuclease expression in bacteria may be achieved by the combinatory use of the FLP site specific recombination system in conjunction with a temperature sensitive repressor of FLP gene expression. The thermolability of FLP, and lack of activity at temperatures above 39° C. may be exploited in this sense to exert dual control over FLP activity where the promoter region of the endonuclease gene contains flanking FRT sites. FLP could be used to invert the promoter region to induce endonuclease expression, whilst expression is absent in the presence of a reversed promoter (Buchholz, et al, 1996, Nucleic Acids Res 24 (21): 4256-62).

In one embodiment, the or each gene for the endonuclease is placed under the same transcriptional control as the enzyme which causes recombination, preferably one which causes intramolecular recombination in both circular and linear DNA and most especially Cre recombinase. A preferred promoter in this embodiment is the arabinose promoter.

The DNA endonuclease may be a restriction endonuclease and/or an intron-encoded endonuclease. It is preferred if endonucleases with extremely rare recognition sequences are used to as to reduce the chances of the endonuclease destroying the chromosomal DNA of the bacterium. Normally, the longer the recognition site, the rarer it occurs. One suitable endonuclease is the octanucleotide recognising enzyme NotI: the E. coli genome has few sites for this enzyme. Alternatively, intron-encoded endonucleases can be used. Preferred such enzymes are those which have recognition sites ranging between 15 bp for I-PpoI and 37 bp for I-TevI, as there is no chance that such a site will occur randomly in the E. coli genome. Enzymes which recognise larger sites may also be used. Examples of suitable enzymes include those described in the following. Monteilhet et al Nucleic Acids Research. 2000. 28(5):1245-1251; Flick et al. Nature. 1998. 394(6688): 96-101; Jurica et al Mol. Cell. 1998. 2(4):469-76; Elde et al Eur. J. Biochem. 1999. 259(1-2): 281-288; Mueller et al EMBO J. 1995. 14(22): 5724-5735.

In the fourth and fifth aspects of the invention, the Cre/lox system described above can be used for the site-specific DNA recombination (Sadowski. J Bacteriol 1986; 165 (2): 341-7; McCulloch et al Embo J 1994; 13 (8): 1844-55). Alternatively, the yeast FLP gene (Andrews et al Basic Life Sci 1986; 40: 407-248; Andrews et al Cell 1985; 40 (4): 795-803), integrases of different bacteriophages, resolvases (Garnier et al. Mol Microbiol 1987; 1(3): 371-6), and invertases (van de Putte & Goosen Trends Genet 1992; 8 (12): 457-62) can be used.

The integration system of Streptomyces bacteriophage ΦQC31 may be used as an alternative to Cre-loxP recombination for minicircle vector DNA production. The integration system of phage ΦC31 comprises the enzyme integrase and two DNA sites: attachment P (attP) and attachment B (attB). The minimal sizes of attP and attB are 39 bp and 34 bp respectively (Groth et al (2000) Proc Natl Acad Sci USA. 97: 5995-6000). Recombination catalysed by ΦC31 integrase is unidirectional and therefore it can offer a benefit of increased minicircle yield compared to the Cre-loxP system. In a further aspect, the present invention provides a method for the production of a minicircle, which method comprises providing a plasmid which has a DNA sequence flanked by attP and attB sites; and exposing the plasmid to ΦC31 integrase, thereby to form a minicircle comprising the DNA sequence and a miniplasmid comprising the remainder of the plasmid.

In order to test the efficacy of integration system of phage ΦC31, the following plasmids can be constructed:
1. pBAD75Int which contains the ΦC31 integrase gene is under pBAD promoter. This plasmid confers chloramphenicol (Cm) resistance and has temperature sensitive origin of replication. The araC-pBAD-Int expression cassette is flanked by arms of homology to Escherichia coli lacZ gene;
2. Plasmids pDATT1 and pDATT2 which contain minimal attP site (39 bp) and minimal attB site (34 bp) separated by multicloning site sequence. The plasmids confer Cm-resistance.
3. Plasmid pDATTluc which is a derivative of pDATT2 and contains the luciferase expression cassette between the two attachment sites. The plasmid confers Cm-resistance.
4. Plasmid pDATT-Km which is a bireplicon plasmid. It is a derivative of pDATT2 and contains the insert of plasmid pDS-Red1-N1 between the attachment sites. It confers Cm-resistance and kanamycin (Km) resistance.

Integrase plasmid pBAD75Int and attachment plasmids pDATT1, pDATT2 and pDATTluc have the same Cm-resistance marker. Therefore it is not possible to combine these plasmids to perform a functional test of the integrase activity. Thus, the attachment plasmid pDATT-Km is constructed and introduced into the strain DH10B pBAD75Int using selection by Km-resistance.

Unlike Cre, it is not clear whether ΦC31 integrase can perform recombination inside linear DNA molecule. Therefore, expression of the integrase under joint control with the endonuclease in the minicircle DNA producing strain is not preferred and a more complicated regulation may be required to achieve sequential expression of both enzymes.

The miniplasmid can be extracted using known techniques such as are described in Sambrook J., Fritsch E. F., Maniatis T. Molecular cloning. A laboratory manual. Second edition. 1989. Cold Spring Harbor Laboratory Press. Standard plasmid procedures are usually lysozyme, alkali lysis (Birnboim & Doly, (1979), Nucleic Acids search 7:1513), precipitation of bulk of bacterial DNA and purification of plasmid in the supernatant on commercial columns or by CsCl-ethidium bromide (or CsCl-propidium bromide). CsCl-ethidium-bromide (or propidium iodide) buoyant density gradient may be used (Fukuda et al. (1976) J. Virol. 1976, 17(3):776-87. In addition, lithium chloride may be used for DNA purification (Chakrabarti et al (1992). Biotechnol Appl Biochem. 16(2):211-5). AG 50W-X8 resin (Bio-Rad) may be used for removal of propidium iodide and ethidium bromide from DNA solutions (Rodriquez et al (1983) Recombinant DNA Techniques: An Introduction. Addison-Wesley Publishing Co., Reading, Mass., 153-158)

When an endonuclease is used as described herein, especially when generated in situ from recombinant DNA encoding it, for instance as in Example 2, the caesium chloride extraction may not be required because linear DNA will be degraded by exonucleases or endonucleases or may be removed with the rest of bacterial debris during alkaline lysis extraction procedure. This is a significant advantage in terms of bulk manufacture of minicircles.

Minicircles produced in accordance with the present invention may be used for mitochondrial gene therapy, no vectors for which exist. For example, an omithine transcarbamylase gene sequence, modified for mitochondrial translation (sOTC), was constructed for expression within mitochondria (Wheeler, et al. (1996) Gene 169(2), 251-5), but expression could not be shown. A therapeutic gene, such as the sOTC gene, was inserted between two tRNA genes within the entire mouse mitochondrial genome, and cloned into a bacterial plasmid vector for propagation (Wheeler, et al. (1996) Gene 169(2), 251-5; Wheeler, et al. (1997) Gene 198, 203-209), but again expression could not be shown. Due to the rarity of non-coding sequences within mammalian mtDNA, the presence of a bacterial vector is likely to be deleterious to either or all of the processes of mitochondrial RNA splicing, replication and transcription. Elimination of the bacterial vector sequences should both overcome this problem and reduce the size of these vectors, increasing the ease of their introduction into mitochondria.

The invention also provides kits comprising cells of the invention, and suitable growth medium for the cell. The components of the kit may be provided in separate containers or together and may also include suitable instructions for use.

Preferred features of each aspect of the invention are as for each other aspect mutatis mutandis. The prior art documents mentioned herein are incorporated to the fullest extent permitted by law.

When used herein, unless the context dictates otherwise, when reference is made to a bacterium, it is intended to include other types of cells used for expression of heterologous proteins, such as yeast cells. DNA is the preferred nucleic acid useful in the present invention, although other nucleic acids, such as RNA, can be used. Nucleic acid constructs of the invention include plasmids and viruses.

### DESCRIPTION OF THE DRAWINGS

Reference is made to the accompanying drawings as follows:

FIG. 1A and FIG. 1B: Insertion of cre/araC into the chromosomal lacZ locus of MM294 bacteria.

FIG. 1A: The plasmid pBAD75Cre contains the cre/ara expression cassette flanked by areas of homology to the bacterial lacZ gene (ΔlacZ1 and ΔlacZ2). The chromosomal lacZ gene has been represented here by five regions for simplicity of reference: lacZ start region, ΔlacZ1 region, lacZ Mid region, ΔlacZ2 region and finally lacZ end region, all of which make up the complete lacZ gene. Use of the temperature-sensitive plasmid replicon, pSC101^{ts}, permits selection for integration of the entire plasmid into the lacZ locus, by using conditions non-permissive for plasmid growth (44° C.) and selection for white chloramphenicol resistant (Cm) colonies (loss of function of pSC101^{ts} as shown by X). A second recombination (excision) event, removing the bacterial vector sequences, is selected for by propagation at 30° C. permissive for plasmid replication, and selection of white Cm^{r} colonies. The excised plasmid is not capable of lacZ expression because it still lacks the start and end of the lacZ gene. Cm^{r} selection may be dropped for 3 days, resulting in loss of the Cm^{r} plasmid, giving white chloramphenicol sensitive colonies containing the integrated cre/ara cassette.

FIG. 1B. Targeted insertion of the cre/ara cassette was tested by PCR amplification of a 1.9 kb fragment using one primer in the cre gene and another in the chromosomal part of the lacZ gene. Colony 44₂ was the result of the first recombination event to insert the entire pBAD75Cre plasmid into the lacZ gene, and serves here as a positive control. Colonies 218 and 219 are the result of a second recombination (excision) event leaving solely the cre/ara cassette in the chromosome at lacZ. Colony 252blue has resulted in the excision of the entire plasmid and serves as a negative control.

FIG. 2A and FIG. 2B: Minicircle producer constructs.

FIG. 2A. Nuclear luciferase construct and minicircle. Plasmid pNIXluc for constitutive mammalian luciferase expression was constructed by insertion of the CMV/luc cassette from pCIKluc into pDlox3. This construct will form minicircles by the Cre directed excision of bacterial vector sequences at loxP sites. Differential digestion of the resulting products with an enzyme that cuts only in the bacterial vector and not in the expression minicircle, permits purification of supercoiled minicircle from unwanted linearised producer plasmid and excised bacterial vector using cesium chloride density separation gradients. In the case of mitochondrial constructs, NotI was used to digest the bacterial vector, whilst PvuII was used to digest bacterial vector from luciferase constructs for nuclear gene delivery.

FIG. 2B. Mitochondrial constructs for minicircle production. Plasmid pMEV8 was constructed as described in experimental procedures. In order to further reduce the size of mitochondrial constructs, regions of mitochondrial DNA were PCR amplified and cloned (3 regions arrowed in blue), to create pMEV46 (8.7 kb), including the D loop, the 12S and 16S rRNA regions, the sOTC gene and the origin of light chain replication.

FIG. 3A and FIG. 3B: Time course of minicircle production from nuclear and mitochondrial constructs.

FIG. 3A. Cre induction time course for pNIXluc (undigested). MM219Cre cells containing construct pNIXluc (6.5 kb) were grown overnight in LB+0.5% glucose before induction of cre recombinase expression by media exchange to M9 minimal media+0.5% arabinose for 2-24 hours. This results in the appearance of two new supercoiled excision products; bacterial vector pDlox3Δ (3.4 kb) and luciferase minicircle mNIXluc (3.1 kb). The additional bands above 6.5 kb supercoiled probably represent various alternate concatenations (linear, open circular) of the original plasmid pNIXluc, as well as supercoiled concatamers of both pDlox3Δ and mNIXluc (induced lanes only). The best induction times for effective production of minicircle were between 4-6 hours.

FIG. 3B. Cre induction time course for pMEV8 (EcoRI digested). MM219Cre cells containing mitochondrial producer construct pMEV8 were grown overnight in LB+2% glucose, prior to cre recombinase induction in M9 minimal media+0.5% arabinose for 10-150 minutes. All products were digested with EcoRI. Induction of cre was evident from the appearance of bands corresponding to mitochondrial minicircle mMEV8 (15 kb, 2 kb 0.2 kb) in addition to those of pMEV8 (13.9 kb, 4.5 kb, 2 kb, 0.2 kb) as well as a linear excised vector (pDlox1Δ) band at 3.4 kb. Cre induction appears to initiate as soon as 10 minutes after initial media change, is obvious after 60 minutes, and reaches equilibrium at 120-150 minutes.

FIG. 4A and FIG. 4B: Driving the Cre recombinase reaction to completion by the use of mutant loxP sites.

FIG. 4A. A new luciferase expression construct was constructed, identical to pNIXluc but containing mutant loxP sites; with respectively, a left element (LE) (bracketed text) and right element (RE) (mutation in lower case text) mutation in the last 5 base pairs of each site—(pFIXluc).

FIG. 4B. Recombination between a LE loxP and a RE loxP site results in an excised bacterial vector product containing a wild type loxP site (pMlox3Δ) and a minicircle product (pFIXluc) containing a double mutant LE/RE loxP site. Cre recombinase has a slightly reduced affinity for either a LE site or a RE site; however it has a severely compromised recognition of a LE/RE site, which results in a shift in the equilibrium towards minicircle production. In addition, since LE/RE double mutant sites do not easily recombine with each other, the formation of minicircle concatamers should be reduced.

FIG. 5: Comparison of the dynamics of the Cre/loxP interaction for normal or mutant loxP sites. MM219 cells were transformed with either pNIXluc (normal loxP sites) or pFIXluc (mutant loxP sites) and grown overnight in LB+0.5% glucose. Cre induction was carried out in M9 minimal media+0.5% arabinose for 4 hours. All plasmids are undigested. Cre recombination of either producer plasmid (pNIXluc or pFIXluc—each 6.4 kb) produces the respective supercoiled minicircle (mNIXluc or mFIXluc—3.1 kb) as shown, including excised bacterial vector (pDlox3Δ/pMlox3Δ—3.4 kb). Cre recombination of pNIXluc resulted in roughly equal quantities of the three major reaction components—producer plasmid, minicircle and excised vector—(6.5 kb, 3.1 kb, 3.4 kb respectively). However, recombination of pFIXluc (6.4 kb) although not complete, produces a greater quantity of minicircle mFIXluc (3.1 kb) compared to excised bacterial vector (3.4 kb). This is probably due to a reduced ability of Cre to recombine minicircle mFIXluc products with either themselves or the producer plasmid because of the double mutant loxP site in the minicircle. Cesium chloride purified minicircle mFIXluc does show some concatamerisation (6.2 kb—minicircle X2, 9.3 kb—minicircle X3, etc), probably as a result of general recombination from the MM219Cre recA+ strain but most of the minicircle DNA was in the single 3.1 kb supercoiled concatamer form. Supercoiled mFIXluc minicircle yields from 1 litre of bacterial culture of 0.75 mg were however considerably higher than those of pNIXluc (0.25 mg) from the same culture volume.

FIG. 6A and FIG. 6B: Comparisons of luciferase activity from HeLa cells transfected with liposome/DNA complexes using different minicircle and plasmid constructs.

FIG. 6A. Means of six replicates of luciferase activity following transfection with DNA/lipofectamine complexes (ratio at 20 ug lipofectamine/μg DNA). Treatment regimes of mole:mole ratios with stuffer DNA, weight-weight and mole:mole without stuffer comparisons are given in table 1. Plasmids pFIXluc and pCIKluc gave roughly similar levels of luciferase activity in mole:mole ratios with stuffer, demonstrating similar gene expression and transfection abilities. Minicircle luciferase activity was increased over pFIXluc by 4.5 fold in mole:mole ratios with stuffer DNA, 8.8 fold in weight-weight ratios and 152 fold in mole:mole ratios without stuffer. The first increase demonstrates an intrinsic increase in minicircle transfection ability or gene expression, probably as a result of multimeric concatamers of minicircle. The second shows that the increased (2.1 fold) number of transcriptional units gives a concomitant increase in transgene activity without changing lipofectamine quantities. The final figure demonstrates the cytotoxicity of lipofectamine, as reduced quantities of this reagent with minicircle result in vastly increased transfection efficiency. Although these figures are adjusted for total protein quantities per measurement, cell cytotoxicity will still result in reduced gene expression from the surviving cells.

FIG. 6B. Log₁₀ transforming data from luciferase activity provides a method for satisfying the conditions required to perform analysis of variance (normality of data and equal variances). In this case F is extremely significant at p≦1.7×10⁻¹⁸. We have then used the studentised values of Q to perform a multiple comparisons test between any two pairs of means from these values. The resulting bar shows the minimum distance required between any two means for at least 95% confidence in a significant difference. We can see that comparative increases in luciferase activity from minicircle over either pFIXluc or pCIKluc within each treatment are significant at this level (p≦0.05) in all cases.

FIG. 7: Amplification of PvuII gene from Proteus vulgaris ATCC13315. Twenty five PCR cycles were performed using Platinum Pfx polymerase (Invitrogen). The annealing was at 60° C. for 1 minute, the reaction was at 68° C. for 2 minutes, the denaturation was at 94° C. for 15 s. Electrophoresis in 0.7% agarose shows the expected 498 bp PCR product.

FIG. 8: PCR with the plasmid DNA from the 12 L-arabinose sensitive bacterial clones obtained in the experiment to clone gene for PvuII endonuclease. Taq-polymerase and primers PvuF and PvuR were used.

FIG. 9: Uncut plasmid DNA from the 12 L-arabinose sensitive bacterial clones obtained in the experiment to clone the gene for PvuII endonuclease in E. coli. The electrophoresis in 0.6% agarose shows heterogeneity in the electrophoretic mobility of the isolated plasmids.

FIG. 10A and FIG. 10B: Structural instability of the plasmids from the 12 L-arabinose sensitive bacterial clones obtained in the experiment to clone gene for PvuII endonuclease in E. coli.

FIG. 10A. PCR strategy to study the physical structure of the plasmids obtained in the experiment to clone the gene for PvuII endonuclease. The drawing shows a section of the vector plasmid pBAD75Cre with the presumed insertion of the KpnI-fragment containing the PvuII gene. Primer LACZ-FA is 5′-CATGGTCAGG TCATGGATGA GCAGACG-3′. Primer ARAC-PCR is 5′-CTGCCGGGAT ACTCGTTTAA TGCCCATC-3′. Primer LACZ2 is 5′-GCGCCACCAT CCAGTGCAGG AGCTCG-3′. There are no rearrangements in araC gene of all plasmids from L-arabinose sensitive clones as shown by PCR with primers LACZ-FA and ARAC-PCR. Thirty PCR cycles were performed using Taq polymerase. The annealing was at 62° C. for 40 s, the reaction was at 72° C. initially for 2 minutes with extension 2 s per cycle, the denaturation was at 93° C. for 30 s.

FIG. 10B. Long distance PCR with primers LACZ-FA and LACZ2 shows different rearrangements in the pBAD promoter region of the plasmids from the L-arabinose sensitive clones 1, 2, 3, 4, 6 and 11. The PCR was performed using the “Expand Long Template PCR system” (Roche) according to the manufacturer's instructions. Absence of the PCR product with the plasmid from clone 6 (pPvuII-6) can indicate the loss or inversion of the plasmid homology to the primer LACZ2.

FIG. 11A-11F: Construction of minicircle DNA producing plasmid pMLOX6-Sp. KF-Klenow fragment of Escherichia coli Polymerase I. Known unique sites are underlined. Used sites are shown in bold.

FIG. 11A, FIG. 11B and FIG. 11C: Construction of pMLOX5 from pMLOX3. Ligation of the KF-treated HindII-ends generates NheI site. Thus NheI-digestion of the ligation mixture was used to destroy the self-ligated vector to facilitate selection of pMLOX5.

FIG. 11D and FIG. 11E: Construction of pMLOX6-Sp from pMLOX5.

FIG. 12: Increased proportion of the minicircle DNA to other products of Cre-recombination after L-arabinose induction of Cre and PvuII in the strain MM219Cre pPvuII-6 pMLOX6-Sp. The strain MM219Cre pMLOX6-Sp was used as a control. Supercoiled minicircle is easily recognisable among the Cre-recombination products of pMLOX6-Sp because the 2 kb minicircle has the highest electrophoretic mobility.

FIG. 13A. Some types of site-specific recombination catalysed by the bacteriophage ΦC31 integrase (Int)

FIG. 13B. Intramolecular recombination catalyzed by the bacteriophage ΦC31 integrase in vitro. Intermolecular recombination between attB plasmid pRT602 and attP plasmid pRT700 (Thorpe et al., 2000, Mol Microbiol, 38(2): 323-41) was used as a control. The substrate DNA was incubated with 0.5 units and 0.125 units of the enzyme for 1 hr at 30° C., extracted with phenol, precipitated, dissolved and digested with appropriate restriction endonucleases. The description of the gel and the predicted sizes of the cut substrates and recombination products are indicated in the table below.

| | | | | Co-integrate |
|---|---|---|---|---|
| Lane | Description | Element 1 | Element 2 | form |
| 1 | 1 kb ladder (Gibco) | | | |
| 2 | pDATT2Km | 4.7 kb | 3.4 kb | 2.4 kb + 5.7 kb |
| | XmnI + BglII | | | |
| | (no integrase) | | | |
| 3 | pDATT2Km | 4.7 kb | 3.4 kb | 2.4 kb + 5.7 kb |
| | Int (0.5) | | | |
| | XmnI + BglII | | | |
| 4 | pDATT2Km | 4.7 kb | 3.4 kb | 2.4 kb + 5.7 kb |
| | Int (0.125) | | | |
| | XmnI + BglII | | | |
| 5 | pDATT2luc | 3.1 kb | 3.4 kb | 1.1 kb + 5.4 kb |
| | XmnI + BglII | | | |
| | (no integrase) | | | |
| 6 | pDATT2luc | 3.1 kb | 3.4 kb | 1.1 kb + 5.4 kb |
| | Int (0.5) | | | |
| | XmnI + BglII | | | |
| 7 | pDATT2luc | 3.1 kb | 3.4 kb | 1.1 kb + 5.4 kb |
| | Int (0.125) | | | |
| | XmnI + BglII | | | |
| 8 | pDATT2Km | 4.7 kb | 3.4 kb | 2.0 kb + 6.1 kb |
| | AflII + XmnI | | | |
| | (no integrase) | | | |
| 9 | pDATT2Km | 4.7 kb | 3.4 kb | 2.0 kb + 6.1 kb |
| | AflII | | | |
| | Int (0.5) | | | |
| | XmnI | | | |
| 10 | pDATT2Km | 4.7 kb | 3.4 kb | 2.0 kb + 6.1 kb |
| | AflII | | | |
| | Int (0.125) | | | |
| | XmnI | | | |
| 11 | pRT602 | 3.0 kb | 2.5 kb | 0.5 kb + 5.0 kb |
| | pRT700 | | | |
| | AatII | | | |
| | (no integrase) | | | |
| 12 | pRT602 | 3.0 kb | 2.5 kb | 0.5 kb + 5.0 kb |
| | pRT700 | | | |
| | Int (0.5) | | | |
| | AatII | | | |
| 13 | pRT602 | 3.0 kb | 2.5 kb | 0.5 kb + 5.0 kb |
| | pRT700 | | | |
| | Int (0.125) | | | |
| | AatII | | | |
| 14 | pDATT1 | | 3.4 kb | |
| | XhoI | | (size marker) | |
| 15 | pDS-RedN1 | 4.7 kb | | |
| | XhoI | (size marker) | | |
| 16 | pDATT2luc | 3.1 kb | 3.4 kb | |
| | BglII + BamHI | (size marker) | (size marker) | |
| 17 | 1 kb ladder (Gibco) | | | |

### DETAILED DESCRIPTION OF THE INVENTION

The invention will now be described with reference to the following non-limiting examples.

### Example 1

Experimental Procedures

Plasmids, Strains and Oligonucleotides

Plasmid pBC SK(+) was purchased from Stratagene, Plasmid pDSRed1-N1 was purchased from Clontech. Plasmids p705Cre, pBAD33Cre and pSVpAX1, as well as bacterial strain MM294, were kind gifts from Dr. F. Buchholz and Dr. A. F. Stewart EMBL). Plasmid pCIKluc was a gift from Dr. D. Gill and Dr. S. Hyde (Oxford University). Mitochondrial plasmids pRSmtOTCAP^{r}, and pRSmtJMC, were made as previously described (Bigger et al, (2000) Anal Biochem 277(2), 236-242. Oligonucleotides (Genosys) DLOX 5′-GGAATTCATA ACTTCGTATA ATGTATGCTA TACGAAGTA TTAATCTCGA GTAATAACTT CGTATAATGT ATGCTATACG AAGTATGGT ACCGCGCCCG-3′ and REVDL 5′-CGGGCGCGGT ACCATAACT-3′ were used to synthesise a DNA fragment with two loxP sites to ultimately create plasmid pDlox3, as well as to reconstruct the ND5/ND6 junction to create pDlox1. Oligonucleotides LINK1 5′-TCGAGTCGAC TCTAGAGGAT CCGAGCTCCC CGGGAAGCTT CTGCAGT-3′ and LINK2 5′-TCGAACTGCA GAAGCTTCCC GGGGAGCTCG GATCCTCTAG AGTCGAC-3′ were used to create a polylinker sequence for the plasmid pDlox3. Oligonucleotides LoxF 5′-CTCGAATTCA TAACTTCGTA TAGCATACAT TATACGAACG GTACTCGAGT ACCGTTCGTA TAGCATACAT TATACGAAGT TATGGTACCA AAAA-3′ and LoxR5′-TTTTTGGTAC CATAACT-3′ were used to create LE and RE mutant loxP sites to ultimately create construct pFIX. Primers NsiICre 5′-GTGAATGATG TAGCCGTCAA G-3′ (homologous to a sequence in the cre gene) and CreIntFwd 5′-CCATGATTAC GGATTCAC-3′ (homologous to nucleotides 2-18 of the chromosomal lacZ gene) were used to amplify a 1.9 kb region, demonstrating insertion of the cre-araC cassette into the bacterial genome.

All constructs were sequenced over the insertion regions and gene expression regions including loxP sites using the Big Dye kit (Perkin Elmer), on a Perkin Elmer 377 sequencing apparatus.

Construction of the pBAD75Ce Targeting Plasmid

Plasmid p705Cre was adapted by the excision of part of the cre gene, the promoter and most of the CI 857 temperature sensitive repressor, at NsiI/RsrII sites. This 583 bp fragment was then replaced with the 1624 bp control regions from pBAD33Cre, including the same part of the cre gene, the BAD promoter, and the araC regulator, also using NsiI/RsrII sites to create pBAD75Cre.

Construction of the MM219Cre Strain

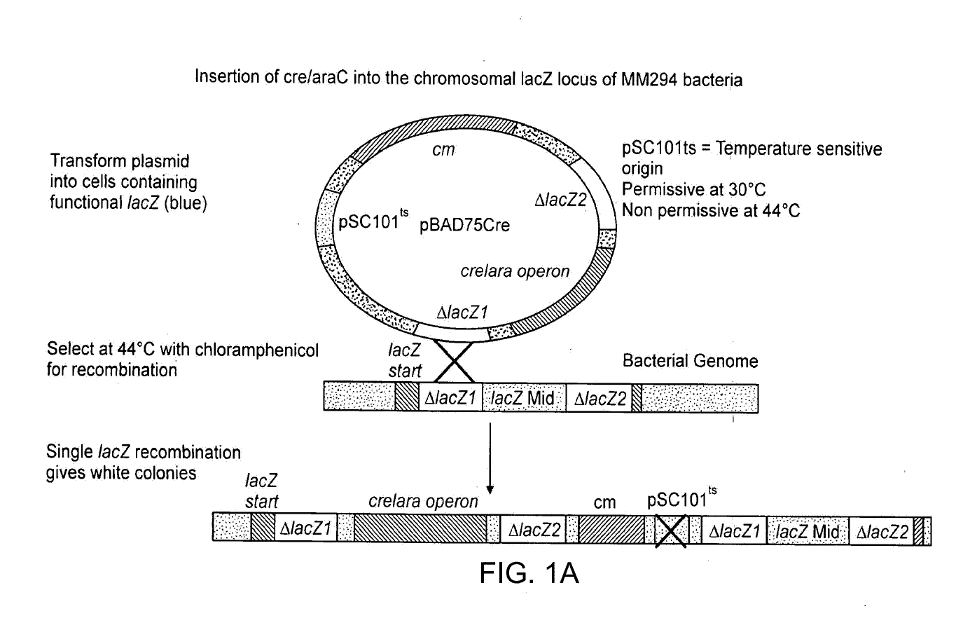

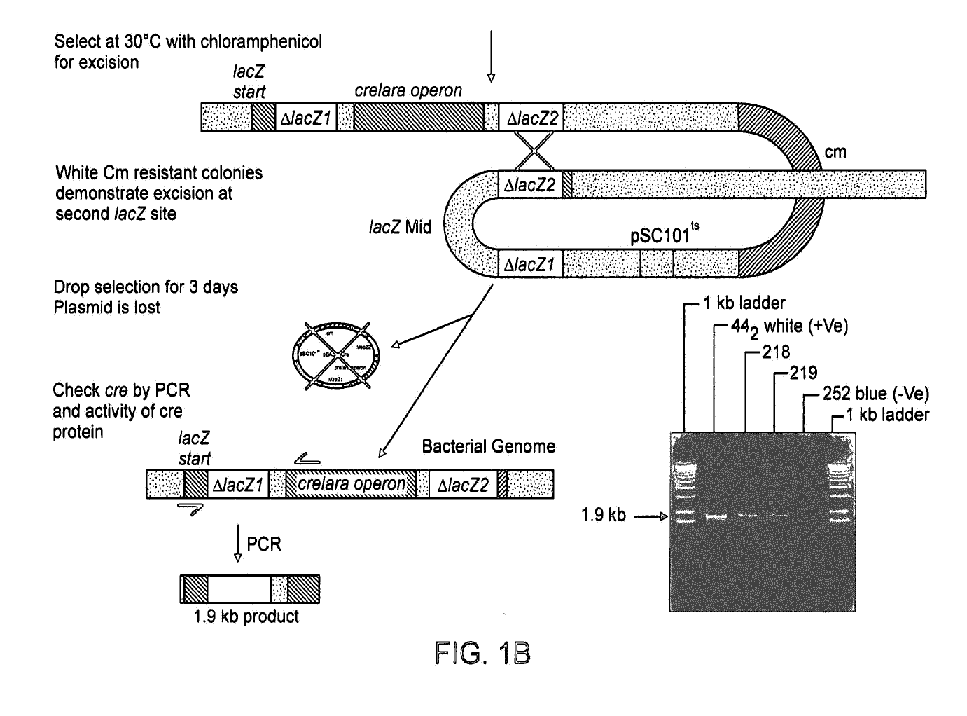

The recombination competent (recA+) bacterial strain MM294 was transformed with pBAD75Cre, and the cre/araC cassette inserted into the bacterial lacZ gene using the targeting method of Hamilton et al (Hamilton, et al. (1989) J Bacteriol 171(9), 4617-22) (FIG. 1A and FIG. 1B), to produce strain MM219Cre.

Construction of pDlox1 and pDlox3 Dual loxP Plasmids

The SacI site was removed from pBC SK(+) by SacI digestion, filling-in with Klenow (Gibco BRL) and religation. Two loxP sites were inserted into the resulting pBC SK-SacI⁰ plasmid by annealing DLOX and REVDL oligonucleotides, endfilling with Klenow, digestion of both the fragment and the plasmid by EcoRI/KpnI and subsequent ligation to create pDlox1. The polylinker was removed by XbaI/PstI digestion, endfilled with Klenow, and ligated to form pDlox2. Then a new polylinker formed by the annealing of LINK1 and LINK2 was introduced between the loxP sites of pDlox2 at XhoI to create pDlox3.

Construction of pNIXluc and Mutant loxP Containing pFIXluc Nuclear Plasmids

Plasmid pNIXluc was created by the insertion of the BamHI/BglII luciferase cassette from pCIKluc, into the BamHI site of pDlox3.

Dual mutant loxP sites (LE and RE) were introduced into pBCSK+ by annealing LoxF and LoxR oligos, filled-in with Pfx polymerase (Gibco BRL), further digestion with EcoRI/KpnI, and ligation to create pMlox1. The unwanted polylinker was removed from pMlox1 by PstI/XbaI digestion, Klenow treatment and self ligation to produce pMlox2. A replacement polylinker was added within the loxP sites by the insertion of the entire pDSRed1-N1 plasmid at XhoI (pMlox3), before removal of the remainder of pDSRed1-N1, excluding the polylinker, by BamHI/NheI digestion, endfilling using Klenow and subsequent ligation to create pFIX. Plasmid pFIXluc was created by the replacement of the pDSRed1-N1 BamHI/BglII fragment from pMlox3 with the BamHI/BglII luciferase cassette from pCIKluc.

Construction of pMEV8 pMEV46 pMEV88 Mitochondrial Plasmids

Construct pMEV8 was made by the insertion of pDlox1 into the unique XhoI site of pRSmtOTCAP^{r}ΔXhoI (Bigger, et al. (2000) Anal Biochem 277(2), 236-2). The ampicillin resistant vector pRS316 was removed from this construct by digestion with SacI and religation to form pMEV8.

Construct pMEV46 was formed by exchange of pRS406 with pDlox3 at the BamHI site of pRSmtJMC (Bigger, et al. (2000) Anal Biochem 277(2), 236-2). Construct pMEV88 was constructed by the deletion of the 16S and most of the 12S rRNA genes at the Klenow filled BlpI/SnaBI sites of pMEV46.

Minicircle Production and Purification

Electrocompetent MM219Cre cells (25 μl) were electro-transformed (BioRad Gene pulser) according to manufacturers instructions, with the appropriate minicircle producer plasmids. Transformed cells were allowed to recover for 1 hour in Luria Bertani media (LB) containing 1% glucose, before plating on LB 1% glucose containing 30 μg/μl chloramphenicol (Cm). Selected colonies were amplified in LB 1% glucose, Cm and frozen in 20% v/v glycerol. Transformed cells containing a minicircle producer plasmid were grown as a 5 ml starter culture overnight at 37° C. in LB 1% glucose with Cm, before inoculation of 500 ml flasks. The most successful growth and cre induction conditions were as follows:

Technique 1

Cells were grown overnight in a shaking incubator at 37° C. in modified M9 minimal media (with the addition of 0.2% yeast extract) (Difco) supplemented with 0.2% glucose, and 30 μg/μl Cm (Sigma Aldrich). Cells were pelleted at 5000 rpm for 10 minutes before resuspension in 1 volume of modified M9 minimal media. After washing, cells were re-pelleted at 5000 rpm and resuspended in the same volume of cre induction media (modified M9 minimal media supplemented with 0.5% L-arabinose (Sigma Aldrich)), and further grown in a shaking incubator at 37° C. for 2-4 hours.

Technique 2

Cells were grown overnight at 37° C. in LB supplemented with 0.5% glucose, and 30 μg/μl of Cm. Cells were pelleted at 5000 rpm for 10 minutes before resuspension in 1 volume of M9 minimal media. After washing, cells were re-pelleted at 5000 rpm and resuspended in the same volume of cre induction media (M9 minimal media supplemented with 0.5% L-arabinose) and further grown in a shaking incubator at 37° C. for 4-6 hours.

1 litre of cells were treated in 5 mg/ml lysosyme in 40 ml Solution I (50 mM glucose, 25 mM Tris.Cl pH 8.0, 10 mM EDTA), followed by lysis in 80 ml (0.2N NaOH, 1% SDS) and finally neutralised in 60 ml 3M potassium acetate (pH 4.8). The cleared supernatant was isopropanol precipitated and the resulting DNA solution further purified by RNA precipitation in 6M lithium chloride, RNAse treatment and phenol/chloroform extraction (Tolmachov, (1990) Biotekhnologiya 1, 25). This technique provides very high yields of DNA per litre of culture (^{˜}10 mg).

The resulting pool of DNA products, producer plasmid and excised bacterial vector were cut with the triple cutting PvuII for luciferase plasmids and with NotI for mitochondrial plasmids. Undigested supercoiled minicircle could then be density separated from linear producer plasmid and excised bacterial vector on a cesium chloride gradient using the intercalating agent ethidium bromide (Radloff, et al. (1967) Biochemistry 57, 1514-1521), or more effectively, propidium iodide. Removal of cesium chloride was achieved by dilution in 3 volumes of water, ethanol precipitation and two washes in 70% ethanol (Sambrook, et al. (1989) Molecular cloning: A Laboratory Manual, Cold Spring Harbour Laboratory, Cold spring Harbour N.Y.). Minicircle DNA was run through cation exchange columns AG50W-X8 (BioRad) to remove ethidium bromide or propidium iodide according to manufacturers instructions in order to achieve maximal DNA yield from columns.

Transfection of Mammalian Cells with Minicircles and Control Plasmids

2×10⁵ cells were seeded into a 24 well tissue culture plate in 1 ml of growth medium (DMEM (Life Technologies)+10% (v/v) fetal calf serum (FCS)) and incubated at 37° C. until 50-80% confluent (approximately 16 hours). 0.24-0.5 μg DNA in 100 μl OPTIMEM media (Life Technologies) was complexed to Lipofectamine (Gibco BRL) in 100 μl OPTIMEM media (2 mg/ml) in the ratio of 10 μl lipofectamine/μg DNA, according to manufacturers instructions. In order to obtain 6 replicates per treatment, this reaction was appropriately scaled-up and the DNA-liposome complex allowed to form at 37° C. for 20 minutes. Cells were washed once in OPTIMEM and a 200 μl reaction volume of complexed DNA in OPTIMEM was then overlaid onto the cells in each well. 4 hours later 1 ml DMEM containing 10% (v/v) FCS was added and the incubation continued at 37° C. 24 hours after the start of transfection, the media was exchanged (DMEM+FCS) and 24 hours following this, cells were harvested and transgene activity measured.

Measurement of Relative Luciferase Activity and Statistical Analysis

Luciferase activity was measured using the Luciferase Reporter Gene Assay kit (Roche pharmaceuticals) on a Lucy1 luminometer (Anthos, Gibco Life Technologies, UK) according to manufacturers instructions. The total protein per measurement was determined in a calorimetric assay using the Micro BCA Protein Assay Reagent kit (Pierce, Rockford, Ill., USA) according to manufacturers instructions. Relative light units of luciferase activity per minute per measurement were then adjusted to that obtained for 1 mg of total protein per measurement.

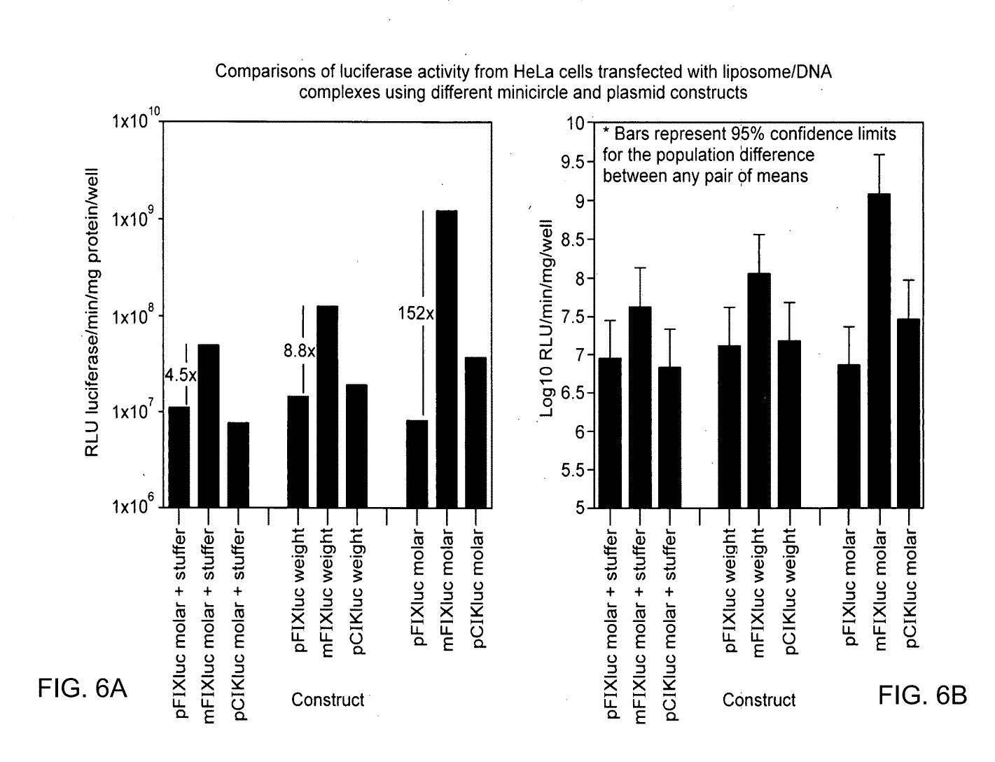

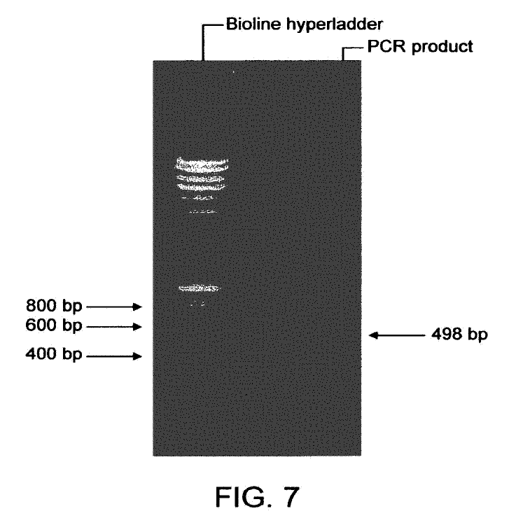

Significance tests were based on the mean from 6 replicates for each assay. In order to satisfy requirements for analysis of variance (ANOVA), raw data was transformed by taking the Log₁₀ of each figure (FIG. 6B). This results in data which are relatively normally distributed (Shapiro-Wilk test) within treatments, with more equal treatment variances.

We have used the analysis of variance to determine the pooled variance for the 9 treatments and subsequently used a method for multiple comparisons based on the studentised range (Q) between means, which is considerably more stringent than either 95% confidence intervals based on 1.96 (standard error), or the least significant difference test. Given that all sample sizes are equal between compared treatments (6 replicates each), this determines a critical value (w) for the difference between the largest and the smallest sample means and applies this to the whole experimental set to obtain a 95% confidence interval between any pair of means. The value of the Q method is such that when comparing all of the differences between means in this manner over a large number of treatments, the probability that no erroneous claims of significance are made is ≧95%.

Results

Creation of a Bacterial Strain Expressing Cre Recombinase Under the Control of the Arabinose Regulon

The vector pBAD33Cre, a direct derivative of the pBAD33 expression vector (Guzman, et al. (1995) J Bacteiol 177(14), 4121-30) containing the arabinose control regulon (araC), was modified to create a new cre recombinase expressing bacterial strain (FIG. 1A and FIG. 1B).

The plasmid, p705Cre, which also expresses cre recombinase, has a leaky λP^{R} based expression cassette flanked by regions of homology to the bacterial lacZ gene, permitting targeted insertion into the bacterial genome by homologous recombination.

Replacement of the cre expression cassette in p705Cre with the cre/araC expression cassette from pBAD33Cre resulted in the creation of a targeting plasmid pBAD75Cre.

Controlled cre expression from this new plasmid was tested by co-transforming bacteria with pBAD75Cre and the Cre reporter construct pSVpaX1, which uses a convenient lacZ based assay for Cre activity (Buchholz, et al. (1996) Nucleic Acids Res 24(15), 3118-9). Growth on LB media containing arabinose led to Cre mediated excision of a 1.1 kb segment from this plasmid and lacZ inactivation giving white colonies. Growth on media containing glucose led to no Cre mediated excision, thus leaving the lacZ gene intact and resulting solely in blue colonies (not shown). This provides good evidence that plasmid based cre expression from the arabinose regulon is absent on growth in glucose containing media, whilst growth in arabinose containing media (in the absence of glucose) results in successful cre expression.

Targeted cre/araC insertion into the recA+ bacterial strain MM294 using pBAD75Cre was achieved by successive rounds of targeted recombination and excision at the lacZ chromosomal locus and the use of the temperature sensitive plasmid replicon pSC101^{ts} (Hamilton, et al. (1989) J Bacteriol 171(9), 4617-22) (FIG. 1A and FIG. 1B).

A PCR based assay was used to determine successful targeted cre/araC insertion into the lacZ gene (FIG. 1B inset) thus creating strain MM219Cre (F⁻X⁻ supE44 endA1 thi-1 hsdR17 lacZ::araC-Cre).

Construction of Minicircle Producer Constructs

To expedite the process of construct manufacture for both nuclear and mitochondrial expression, a multi-cloning plasmid containing dual loxP sites flanking a polylinker (pDlox3) was created from the basic vector pBCSK(+). This plasmid permits easy insertion of expression cassettes or mitochondrial sequences into the polylinker region, to create minicircle producer plasmids.

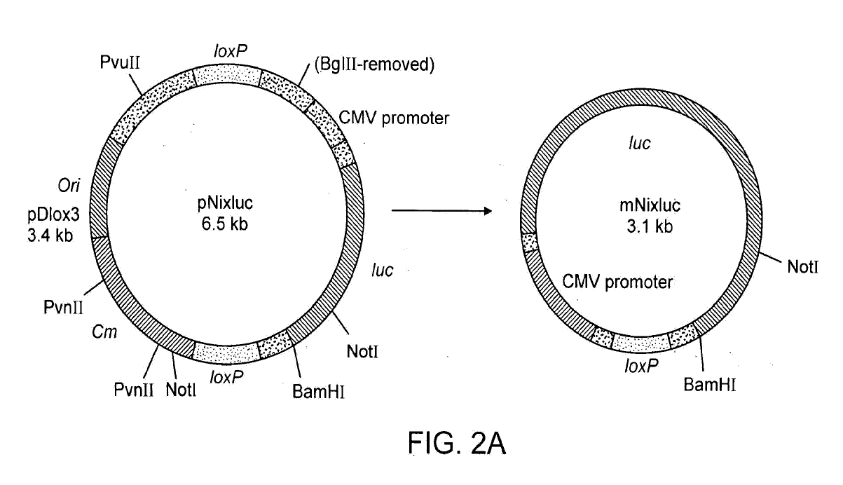

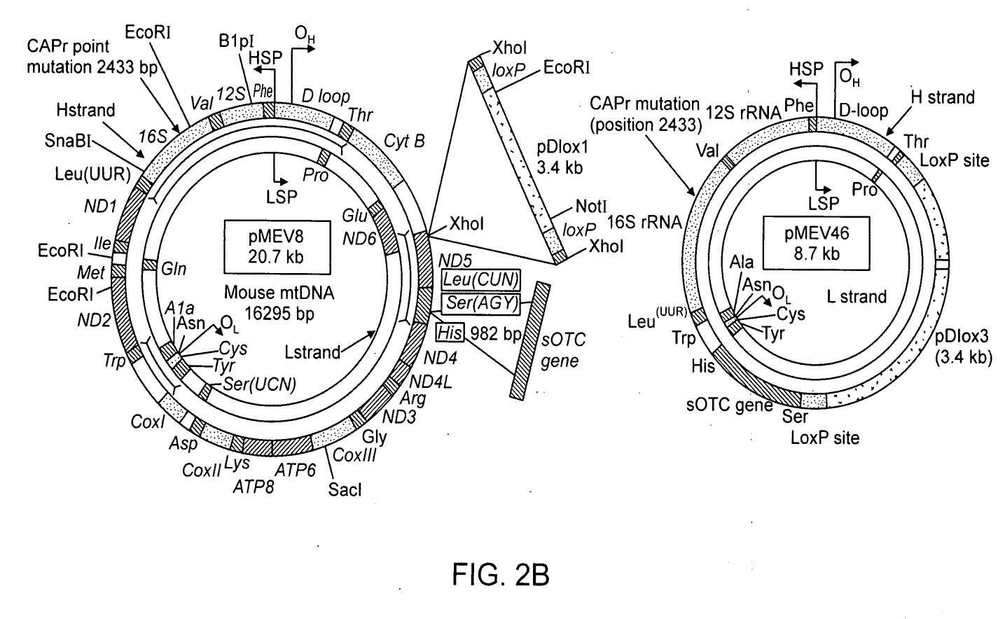

The initial construct for nuclear expression was generated by cloning of the luciferase reporter gene and CMV promoter from the high expression plasmid pCIKluc, into the loxP flanked polylinker of pDlox3. The resulting plasmid pNIXluc contains a minimal sized luciferase expression cassette flanked by loxP sites to permit removal of bacterial sequences by Cre recombination to create mNIXluc minicircle (FIG. 2A).

A 22 kb construct designed for mitochondrial expression based on the insertion of a modified OTC gene between two tRNA sites within the entire mouse mtDNA has previously been created (Wheeler, et al. (1996) Gene 169(2), 251-5; Wheeler, et al. (1997) Gene 198, 203-209; Bigger, et al. (2000)Anal Biochem 277(2), 236-242). This expression construct is difficult to modify due to its instability (Bigger et al, (2000) Anal Biochem 277(2), 236-242) and presents problems for introduction into mitochondria by electroporation due to its large size (Collombet, et al. (1997) Journal of Biological Chemistry 272(8), 5342-5347). In addition, the bacterial vector falls within the mitochondrial gene COXIII, is not easily removable and is likely to abolish mitochondrial gene function.

To ameliorate this situation, the loxP flanked pDlox1 vector was inserted into pRSmtOTCAP^{r} at XhoI and the pRS316 vector removed to create the mitochondrial minicircle producer plasmid pMEV8. This XhoI site in mouse mtDNA is situated in a 14 bp area where the ND5 gene coded on the heavy strand overlaps the terminal coding region of the ND6 gene, oriented in the opposite direction on the light strand. The terminal regions of the ND5 and ND6 genes were reconstructed between the loxP sites of the insertion vector pDlox1 to ensure complete transcription from these genes within pMEV8 (FIG. 2B).

The mitochondrial minicircle resulting from Cre mediated excision of pDlox1Δ from pMEV8 (mMEV8), contains a single 34 bp loxP site flanked by the reconstructed ND5 and ND6 genes. This should minimise the impact of incorrect splicing resulting from the presence of a foreign sequence on transcribed mitochondrial minicircle DNA.

Smaller mitochondrial constructs were also made to permit more efficient DNA transfer into mitochondria, by PCR amplification of key regions of the mitochondrial genome and the sOTC gene (Bigger, et al. (2000) Anal Biochem 277(2), 236-242). Construct pMEV46 consists of the mitochondrial D loop, 12S, 16S rRNA, the origin of light chain replication and several tRNAs, with the loxP flanked pDlox3 inserted at the already artificial Thr/Ser tRNA gene junction (FIG. 2B). An even smaller 6.8 kb derivative, pMEV88 (not shown), lacks most of the 12S and 16S rRNA regions of pMEV46.

As tRNAs are believed to act as cleavage signals within polycistronic mtRNA transcripts (Ojala, et al. (1980) Cell 22(2 Pt 2), 393-403; Ojala, et al. (1981) Nature 290, 470-474), it is anticipated that the 34 bp loxP site will have minimal impact on mitochondrial transcription in these constructs.

All of these minicircle producer constructs are designed to permit excision of the bacterial vector (pDlox1Δ or pDlox3Δ) by Cre recombination to leave solely a 34 bp loxP site within the resulting minicircle constructs (FIG. 2A and FIG. 2B).

Cre Recombinase Activity and Minicircle Production in MM Bacterial Strains

The novel E. coli strain, MM219Cre expresses cre recombinase under tight control of the araC regulon. The AraC protein acts as both a positive and negative regulator of Cre activity. In the presence of arabinose in growth media, transcription from the BAD promoter is turned on; in its absence, transcription proceeds at a very low level. The addition of glucose to growth media, which lowers levels of 3′,5′ cyclic AMP, further down-regulates the catabolite-repressed BAD promoter (Buchholz, et al. (1996) Nucleic Acids Res 24(21), 4256-62; Hirsh & Schleif, (1973) J Mol Biol 80(3), 433-44; Hahn, et al. (1984) J Mol Biol 180(1), 61-72; Kosiba & Schleif, (1982) J Mol Biol 156(1), 53-66).

MM219Cre cells transformed with different minicircle producer plasmids showed effective repression of cre recombinase over a range of media types using varying levels of glucose. Minicircle production and the presence of excised bacterial vector were used as indicators of leaky cre recombinase expression. The three Media types used for bacterial growth in decreasing order of richness were; LB, modified M9 minimal media (containing 0.2% yeast extract) and M9 minimal media, incorporating a range of glucose concentrations from 0.2% to 2%. Rich media (LB) leads to the most rapid growth of both bacteria and plasmid but also results in the exhaustion of glucose. Bacterial growth in M9 minimal media gives comparatively poor bacterial and hence plasmid yields. Initial glucose concentrations higher than about 1% also lead to significant inhibition of bacterial growth, as a result of the Crabtree effect Neidhardt, F. C. (ed) (1987) Esherichia coli and Salmonella typhimurium, cellular and molecular biology Vol. 2. Edited by Ingraham, et al. 2 vols., American Society for Microbiology, Washington D.C.; Aristidou, et al. (1999) Biotechnol Prog 15(1), 140-5; Gschaedler, et al. (1999) Biotechnol Bioeng 63(6), 712-20), although cre induction is still effectively repressed.

The best growth conditions were obtained using levels of 0.2-0.5% glucose with any of the media types, striking a balance between bacterial and thus plasmid replication and down-regulated cre expression.

However, growth of MM219Cre cells containing the largest plasmid, pMEV8 (20.7 kb), in LB 0.2%-0.5% glucose leads to a slight induction of cre, minicircle production and subsequent loss of minicircle during growth. Assuming that there is slight cre expression during bacterial growth using low glucose levels, the potential toxicity of the largest mitochondrial construct may help to induce loss of replication deficient minicircle during plasmid replication under chloramphenicol selection.

Significant minicircle production (and subsequent loss) was not observed using the same low glucose media growth conditions in the case of any other minicircle producer constructs. This is in accordance with data on pBAD expression plasmids for which no significant gene induction effects have been observed under similar low glucose conditions (Guzman, et al. (1995) J Bacteriol 177(14), 4121-30). By changing media type to modified M9 minimal media, glucose levels could be kept low (0.2%) and still effectively down-regulate cre expression using pMEV8, whilst this richer media type permitted increased plasmid yields over that of minimal media alone.

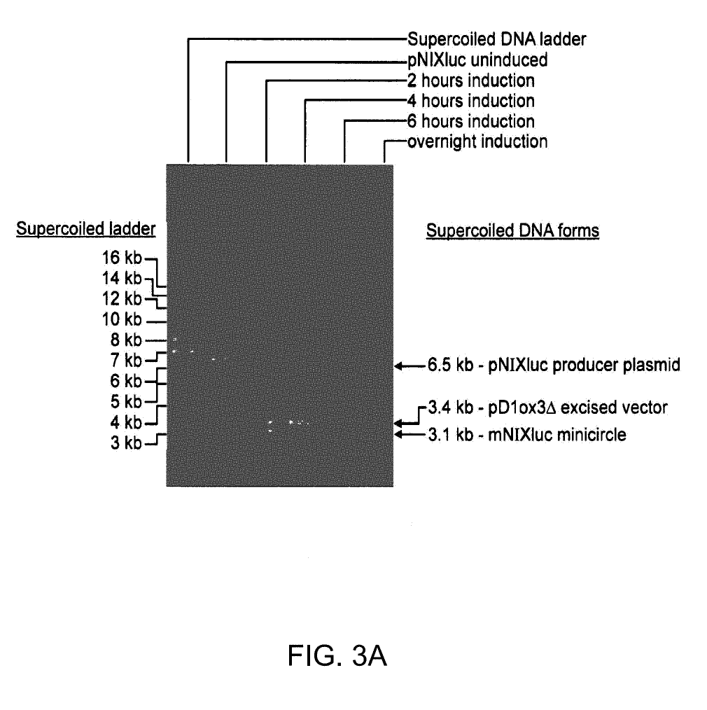

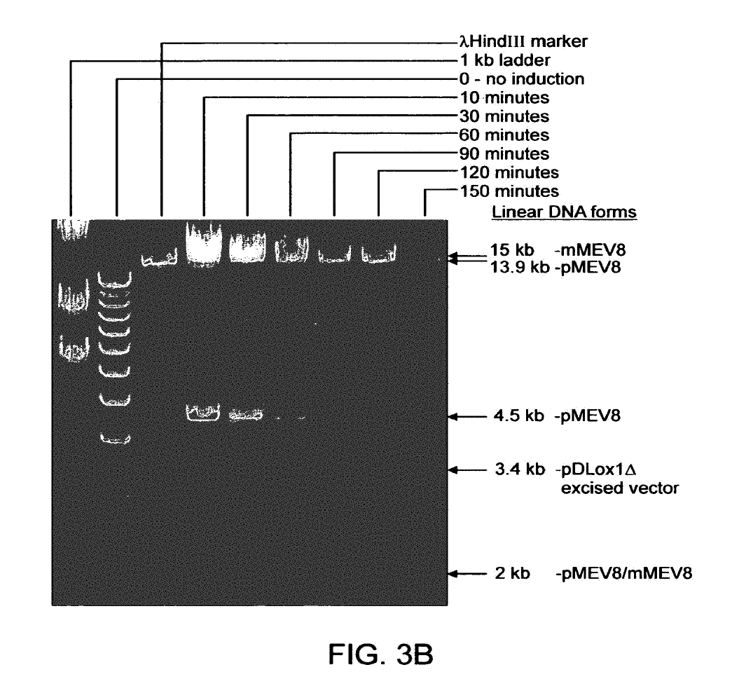

Following bacterial and plasmid growth, induction of cre recombinase and thus minicircle production used either LB, modified M9 minimal media or M9 minimal media, containing levels of arabinose from 0.2%-2%. Arabinose levels had little effect on overall minicircle yields, whilst incubation times of 4-6 hours produced the greatest yields of minicircle from smaller plasmids (FIG. 3A), and shorter incubation times of 2-4 hours for the largest mitochondrial minicircle mMEV8 (FIG. 3B).

The two best techniques for minicircle production were as follows.
Technique 1: Growth in modified minimal media, 0.2% glucose overnight, washing in modified minimal media and induction for 2-6 hours in modified minimal medium containing 0.5% arabinose.
Technique 2: Growth in LB, 0.5% glucose overnight, washing in minimal media and induction for 4-6 hours in minimal media containing 0.5% arabinose.

Following cre recombinase induction, supercoiled minicircle could be purified away from producer plasmid and excised bacterial vector by restriction enzyme digestion of the latter two forms and purification of supercoiled minicircle using a cesium chloride gradient.

Technique 1 was effective for minicircle production from smaller plasmids, with a purified minicircle yield of up to 200 μg/L culture, as well as being the only effective method for producing yields of 40 μg/L culture of minicircle from the large mitochondrial construct pMEV8.

Interestingly, technique 2 produced slightly higher yields of minicircle using smaller plasmids, but was very ineffective for minicircle production from the larger pMEV8 construct, presumably due to minicircle loss during bacterial growth.

Media step down from rich to minimal medium as observed in technique 2 did not seem to reduce cre expression as might be expected, but contrastingly led to a small increase in yields of supercoiled minicircle.

Creation and Testing of a Mutant loxP Containing Construct

Cre recombination may occur between and within minicircle constructs, producer plasmids and bacterial vectors resulting in double, triple etc concatamers as a result of the equilibrium kinetics exhibited by the reaction. Although a significant proportion of the minicircle produced is in the monomeric supercoiled form, reduction of the formation of minicircle concatamers as well as the ability to drive the Cre reaction towards minicircle production, should permit increased yields of minicircle.

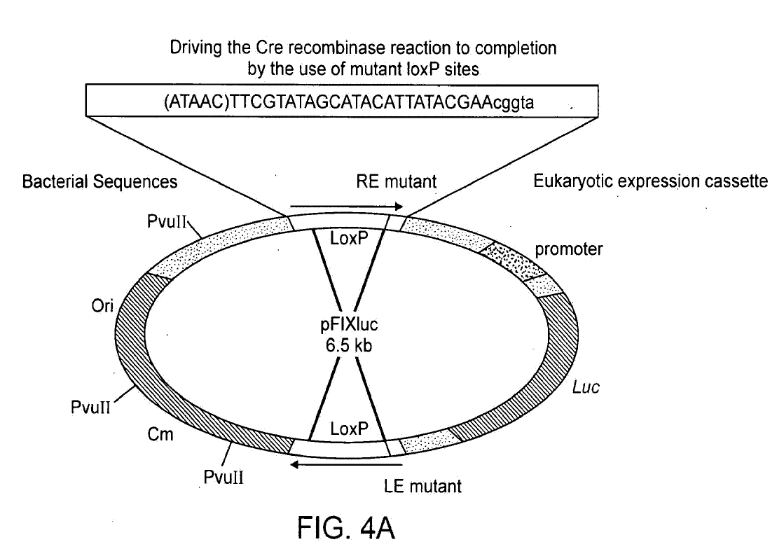

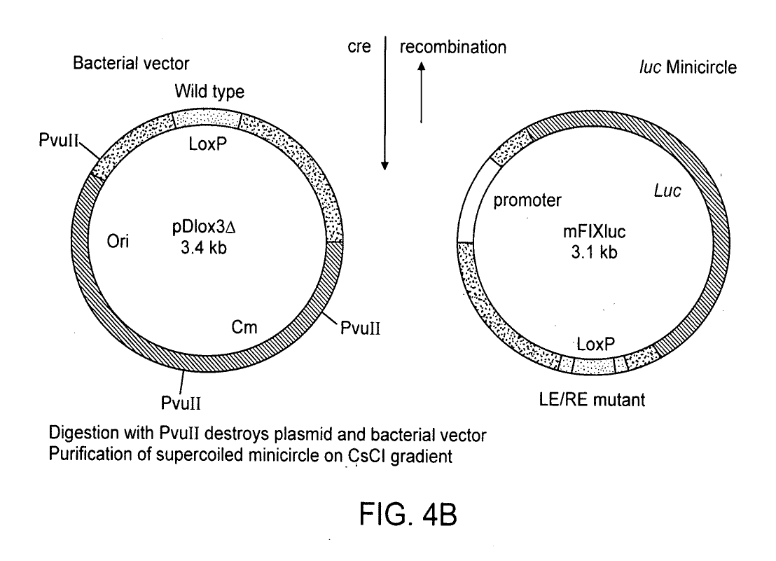

Modification of the terminal 5 nucleotides on one side of the loxP site to create left element (LE) loxP sites, or vice versa to create right element (RE) loxP sites, results in a slightly reduced Cre interaction at these sites (Albert, et al. (1995) Plant J 7(4), 649-59). Modification of both sides of the loxP site to produce LE/RE double mutant loxP sites results in a severely reduced Cre interaction (Albert, et al. (1995) Plant J 7(4), 649-59; Araki, et al. (1997) Nucleic Acids Res 25(4), 868-72). Recombination between two partially mutant loxP sites, one LE and one RE, leads to the production of a double mutant loxP site (LE/RE) and an unmutated wild type loxP site (WT) in the two products (FIG. 4A and FIG. 4B).

Reverse kinetics in this reaction are extremely poor, due to the reduced affinity of Cre for the LE/RE double mutant loxP site. Thus there is a directed drive towards production of an LE/RE site (Albert, et al. (1995) Plant J 7(4), 649-59; Araki, et al. (1997) Nucleic Acids Res 25(4), 868-72).

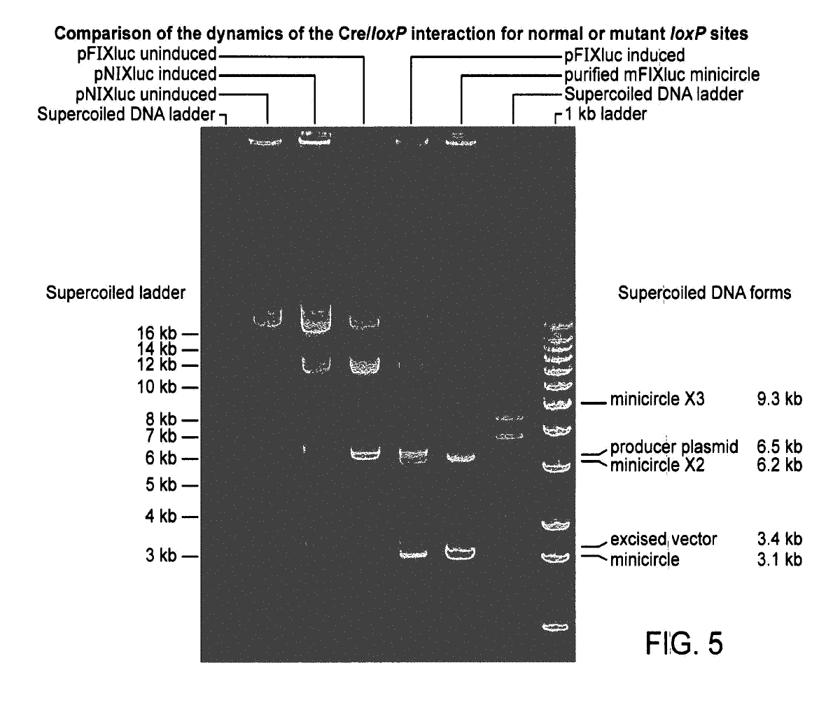

Following this concept, a producer plasmid was created to contain a mutant LE loxP site and a mutant RE loxP site flanking the polylinker region (pFIX). The CMV/luciferase cassette from pCIKluc was inserted between the LE and RE loxP sites to create a new minicircle producer vector pFIXluc. Growth and induction of this producer plasmid pFIXluc using technique 2 resulted in increased levels of monomeric minicircle compared to excised bacterial vector (FIG. 5). Since the construct has been designed such that the minicircle mFIXluc always contains the LE/RE double mutant loxP site, this is probably a result of reduced minicircle concatamerisation and a shift in equilibrium towards minicircle production. This results in a significant increase in overall yield of mFIXluc minicircle over pFIXluc to 300 μg per litre of bacterial culture.

Although maximal obtainable yields of luciferase minicircle measured by spectrophotometry with 260/280 ratios approaching 1.8 were in the region of 5-600 μg/litre of bacterial culture, gel quantification of DNA did not support this data, giving levels approximately 30% lower. Further RNase treatment and phenol/chloroform purification was performed in these cases to obtain agreement between spectrophotometry and gel data. This may have been the result of residual ethidium bromide/propidium iodide skewing spectrophotometry readings, thus emphasising the importance of cross-checking measurement data within batches using gel quantification methods.

The MM219Cre strain is recA+, which probably explains the continued occurrence of supercoiled concatamers of mFIXluc minicircle (FIG. 5), despite the severely compromised Cre interaction at the double mutant loxP sites. Despite this, all mFIXluc concatamer forms could be resolved to the same size (3.1 kb) by enzymatic digestion (not shown), suggesting simple concatamerisation rather than rearrangements. The possibility of large-scale rearrangements and plasmid deletions using MM219Cre seems unlikely, since the large mitochondrial clones pMEV8, and pRSmtOTCAP^{r} can be stably maintained with no observable rearrangements. In further support of this, it has been possible to clone and stably maintain a 150 kb BAC in MM219Cre cells. A recA+ strain may actually encourage stable maintenance of some large constructs, by permitting repair of damaged constructs.

Gene Expression In Vitro Using Luciferase Minicircle Constructs

In order to test the versatility of luciferase expression from our latest nuclear minicircle within mammalian cells, three comparative tests were performed using lipofectamine complexed to DNA to obtain cellular transfection. In each test, luciferase minicircle mFIXluc was compared with its parent plasmid pFIXluc, as well as with the original plasmid from which pFIXluc was derived (pCIKluc), all of which contain a luciferase cassette driven by a CMV promoter. Treatment regimes over 6 replicates for each construct are summarized in Table 1 and FIG. 6A and FIG. 6B.

TABLE 1
Summary of the 3 treatment regimes used to transfect
HeLa cells with DNA constructs using the same ratio
of lipofectamine to DNA in each case (20:1 μg)

| | Treatment per well | | | | |
|---|---|---|---|---|---|
| | PFIXluc | mFIXluc | | pCIKluc | |
| | 6456 bp | 3089 bp | | 5632 bp | |
| Mole:mole with | 0.5 μg | 0.24 | μg | 0.44 | μg |
| stuffer | | | | | |
| DNA | 0 μg stuffer | 0.26 μg stuffer | | 0.06 μg stuffer | |
| (pDlox2 stuffer | | | | | |
| 3409 bp) | | | | | |
| Weight:weight | 0.5 μg | 0.5 | μg | 0.5 | g |
| Mole:mole | 0.5 μg | 0.24 | μg | 0.44 | μg |
| without stuffer | | | | | |

The initial treatment of mole:mole with stuffer compares equal molar ratios of each construct, with the total weight of DNA adjusted to 0.5 μg per well using pDlox2 plasmid. This permits equal levels of lipofectamine to be used for transfection in each case, thus minimising differences resulting from the cytotoxicity of lipofectamine. It should therefore result in equal numbers of transcriptional luciferase units being delivered to cells in each case and is thus the most unbiased comparison of minicircle function. The weight-weight treatment compares equal weights of DNA from each construct. Lipofectamine levels are again equal throughout the treatment but 2.1 times the amount of minicircle luciferase cassettes should be transfected over pFIXluc. Finally the mole:mole without stuffer treatment allows comparison of molar ratios of constructs with variable lipofectamine quantities, whilst keeping the same ratio of lipofectamine to DNA (20:1 μg). Whilst this permits the transfection of equal numbers of transcriptional luciferase units, the variable lipofectamine will give varying results depending on the cytotoxicity of lipofectamine.

FIG. 6A and FIG. 6B demonstrate the results of these 3 treatments using 3 plasmids over six replicates in two different graphical representations. Firstly in FIG. 6A, the means of raw data are presented for each plasmid on a semi-log scale, and secondly in FIG. 6B, the means of log transformed data with 95% confidence limits between any pair of means are presented. The studentised Q test for multiple comparisons, as shown in this case, gives a single bar representing the minimum distance required between any two means to provide 95% confidence in a significant difference. This is in contrast to a 95% confidence interval calculated for an individual mean (1.96×standard error), given by two opposite bars flanking the mean.

Basic luciferase expression from pFIXluc was roughly comparable to that of pCIKluc (its precursor) in the mole:mole+stuffer comparison, suggesting that gene expression and transfection efficiency from the adapted construct pFIXluc is undiminished. In the weight-weight comparison there was a slight but insignificant increase in luciferase activity by pCIKluc over pFIXluc as expected given the increased number of luciferase cassettes theoretically delivered (1.1 fold). Finally, there was a significant increase of pCIKluc luciferase activity over pFIXluc in the mole:mole without stuffer treatment. Despite equal molar quantities of luciferase cassettes transfected per construct the difference is probably due to reduced lipofectamine in the case of pCIKluc producing less cytotoxicity.

Comparisons between the luciferase expression from pFIXluc and mFIXluc were quite conclusive in demonstrating increased minicircle luciferase expression over pFIXluc in all treatments.

Surprisingly, the mole; mole with stuffer treatment produced a 4.5 fold increase in luciferase activity for minicircle over pFIXluc, that was statistically significant (p≦0.05) within the treatment. Theoretically these transfection conditions represent those most likely to give equal levels of transfection in the case of each construct. It should be noted however that although all constructs were produced in the same way, minicircle production involved cre recombination, which produces multimeric concatamers of minicircle, as well as the predominant monomeric form. Multimeric plasmid forms have previously been shown to increase marker gene activity following transfection in vitro (Leahy, et al. (1997) Nucleic Acids Res 25(2), 449-50), perhaps because they provide a more efficient template for nuclear transcription.

Not surprisingly, weight-weight comparisons showed an 8.8 fold increase of minicircle transgene activity over parent plasmid (pFIXluc) (Significant at p<0.05), as expected given that 2.1 times more luciferase cassettes were transfected over the mFIXluc mole:mole with stuffer treatment.

Finally, minicircle luciferase activity over pFIXluc for mole:mole comparisons with no stuffer DNA is vastly increased (152 fold) (Significant at p<0.05). This increase should be treated with caution as it serves to highlight the limitations of lipofectamine as a transfection reagent, where reduced lipofectamine quantities in the case of minicircle transfection cause a huge increase in transgene activity despite equimolar transfection. Indeed transfection of 0.5 μg of DNA into HeLa cells using this reagent at the applied ratio 20:1 is already becoming toxic to these cells. This is also supported by the transfection of pCIKluc using the same treatment and only slightly less lipofectamine, giving a 4.5 fold increase over pFIXluc. Interestingly, transfection comparisons on HeLa cells using either mole:mole with stuffer or weight-weight ratios of 0.25 μg DNA (at lipofectamine levels not toxic to HeLa cells) still show increased minicircle luciferase activity over parental plasmid (not shown).

Discussion

A bacterial strain expressing cre recombinase under the tight control of the araC regulon, which can be used to produce large quantities of DNA minicircle in vivo, has been created. A range of minicircle constructs for both mitochondrial expression of sOTC and for nuclear luciferase expression have also been developed. In addition, both effective and substantially increased luciferase expression from nuclear minicircle constructs over both parental plasmids have been demonstrated.

The mitochondrial minicircles eliminate bacterial sequences which may be able to act specifically as potential mitochondrial origins of replication (Kazakova, et al. (1983) Genetika 19(3), 381-7), or break-points for transcription.

Although the reduced size mitochondrial constructs pMEV46 and pMEV88, made by gene deletion present additional concerns for stability in organello, the minicircle constructs resulting from these producer plasmids (mMEV46, mMEV88) are now of a size which should enable their electroporation into mitochondria (Collombet, et al. (1997) Journal of Biological Chemistry 272(8), 5342-5347.

The nuclear minicircle vectors mNIXluc and mFIXluc clearly possess the advantage of being approximately half the size of their plasmid counterparts. As such, these small constructs demonstrate 4.5 fold increased luciferase activity over their parental plasmid counterparts when transfected on a mole:mole basis (with stuffer DNA) and 8.8 fold increase on a weight-weight basis. The huge increase seen in the mole:mole without stuffer comparison (152 fold) only serves to highlight the versatility of these vectors in reducing the cytotoxic load of DNA/liposome complexes to cells whilst maximising the number of transcriptional units transfected. Indeed by the simple expedient of removing the entire bacterial DNA complement, the CpG content of most of these expression vectors has been reduced by more than 60%. As such, minicircle expression vectors can provide a useful tool for reducing inflammatory responses in non-viral vector delivery in vivo as well as the increased transgene activity already demonstrated in vitro.

### Example 2

The aim of this example is to create a bacterial strain where both Cre-recombinase and PvuII-endonuclease (PvuII) are inducibly expressed, allowing generation of minicircle DNA with concomitant complete or partial elimination of the unwanted recombination products in vivo.

Construction and Selection of pPvuII-6 Plasmid

The chromosomal DNA of Proteus vulgais ATCC13315 was used as the source of a gene encoding the restriction endonuclease PvuII (Athanasiadis et al., (1990) Nucleic Acids Res., 18: 6434; Gingeras et al., (1981) Nucleic Acids Res. 9:4525-4536). High-fidelity Pfx-polymerase (Invitrogen) and primers PvuF 5′-AGCGATGGTA CCATGAGTCA CCCAGATCTA AATAA-3′ and PvuR 5′-TAGGTTGGTA CCTTAGTAAA TCTTTGTCCC ATGTT-3′ were used to obtain the PCR-product containing PvuII gene flanked by KpnI sites (FIG. 6A and FIG. 6B). The PCR-product was digested with KpnI and ligated to the KpnI-digested plasmid pBAD75Cre described in Example 1. A unique KpnI site in pBAD75Cre is located immediately downstream of the Cre gene which is transcribed from the pBAD promoter. The ligation mixture was used to transform electrocompetent DH10B cells (Invitrogen) by electroporation. Expression of PvuII is lethal for bacteria that do not express PvuII-methylase. Therefore, the medium for selection of transformants (LB-agar) was supplemented not only with chloramphenicol (Cm, 30 μg/ml) to select bacterial clones harbouring plasmids but also with glucose (0.5%) to repress the pBAD-promoter. The plates were incubated at 30° C. because pBAD75Cre has a temperature-sensitive origin of replication.

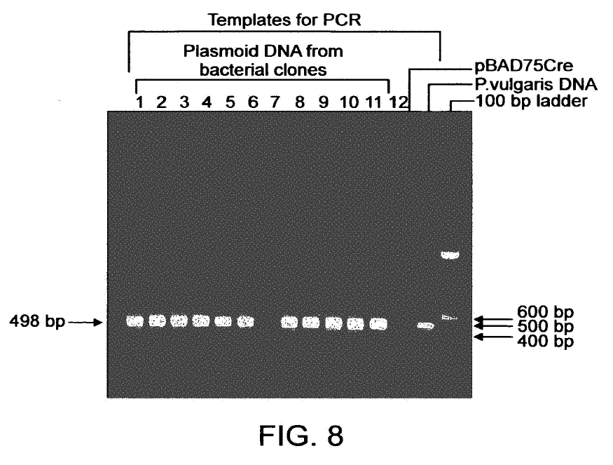

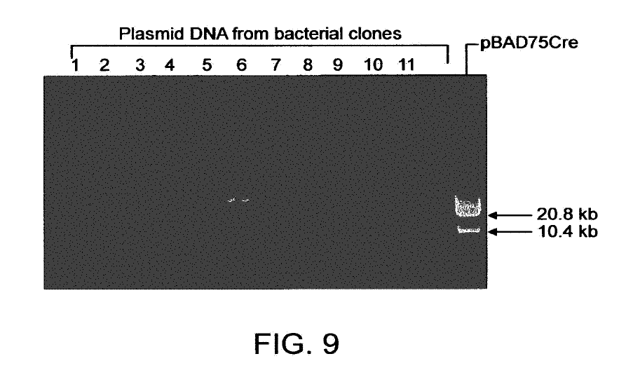

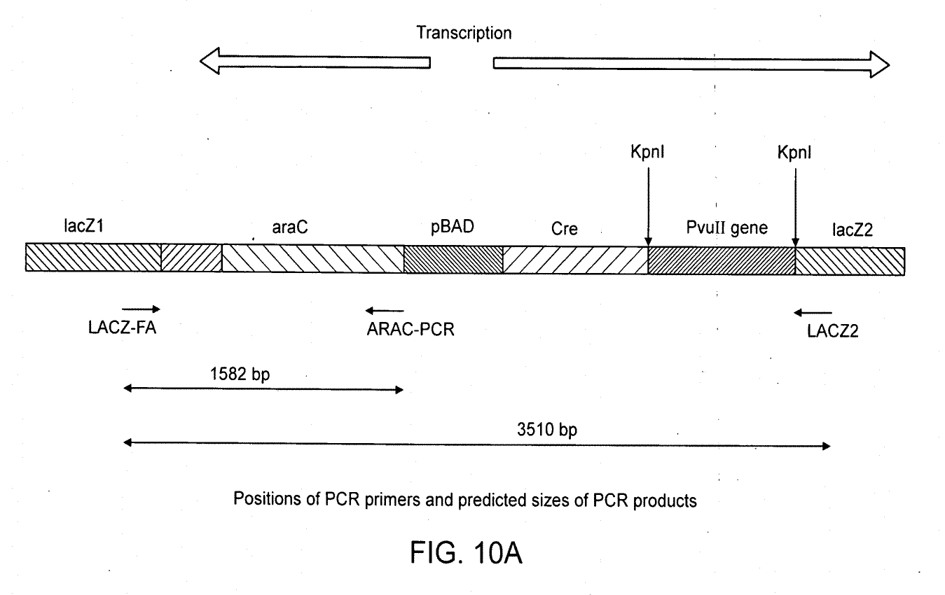

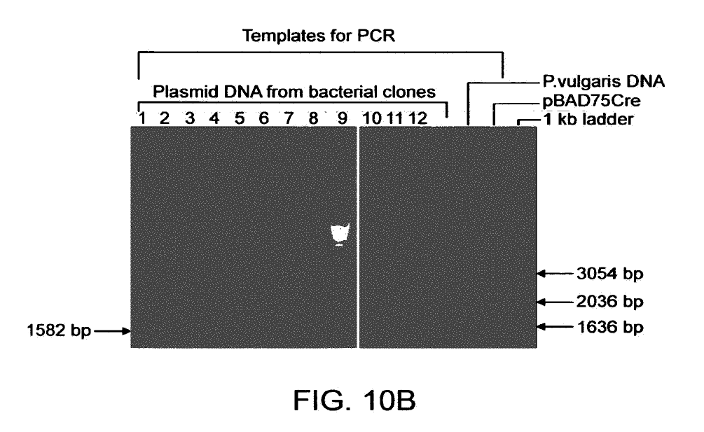

As mentioned above, expression of PvuII gene is lethal for bacteria that do not express PvuII-methylase. Therefore, to find bacterial clones with arabinose inducible expression of the PvuII gene, 600 transformants were screened for their inability to grow on LB agar supplemented with L-arabinose (0.5%) and Cm (30 μg/ml) during 12 hrs at 30° C. Addition of Cm to the screening medium ensures that spontaneously arisen plasmidless bacteria do not grow and obscure the lethal action of PvuII. Twelve clones did not grow on the screening medium. These clones were expanded in 5 ml of LB broth supplemented with Cm (30 μg/ml) and glucose (0.5%) at 30° C. and used to prepare plasmid DNA. The obtained plasmids were used as templates for PCR with primers PvuF and PvuR. 11 out of 12 plasmid DNA preparations contained the entire PvuII gene (FIG. 8), plasmid DNA from clone 11 was negative. Agarose gel analyses of the 12 isolated plasmid DNA preparations indicated variations in plasmid size (FIG. 9). The structural heterogeneity was further confirmed using restriction digestion by EcoRV, HindIII and BspEI (data not shown). Plasmids from clones 1, 5, 8, 10, 12 gave identical restriction patterns; therefore clone 1 was used as a representative of this group in some of the further experiments. Non-equimolar DNA fragments were observed in the restriction digests of plasmid DNA from clones 4 and 9, indicating co-existence of several plasmid species in these strains. A PCR-based strategy was used to study the physical structure of the region of the PvuII gene insertion in the obtained plasmids (FIG. 10A). PCR with primers LACZ-FA and ARAC-PCR revealed no rearrangements in the araC gene in the obtained clones (FIG. 10B). The rearrangements occurred in the pBAD promoter or the Cre gene because long-distance PCR with primers LACZ-FA and LACZ2 showed considerable heterogeneity in size of the entire araC-pBAD-Cre-PvuII module (FIG. 10C).

The obtained results revealed that, even under catabolite repression conditions, the pBAD-promoter does allow occasional transcription of PvuII gene and production of the PvuII enzyme that kills the bacterial cells. Thus, the obtained plasmids with PvuII gene harbour mutations that somehow prevent spontaneous PvuII expression. The majority of such mutations are likely to block or to attenuate expression of PvuII. Rare mutations might confer a tighter control of PvuII expression. Thus, it was assumed that some of the mutant plasmids could provide controlled expression of PvuII. To check this possibility, the plasmids from clone no. 1 (pPvuII-1), no. 3 (pPvuII-3) and no. 6 (pPvuII-6) were introduced into MM219Cre bacteria by electroporation (selection medium contained glucose to repress expression of PvuII). The resulting transformants were streaked on LB agar supplemented with glucose (0.5%) and Cm, and on LB agar supplemented with L-arabinose (0.5%) and Cm.

Growth of the clones harbouring pPvuII-1 and pPvuII-3 was slightly inhibited by L-arabinose, while growth of the clones harbouring pPvuII-6 was strongly inhibited by L-arabinose. The inhibitory effect of L-arabinose was indicative of PvuII expression in the presence of L-arabinose. To be able to monitor expression of both the Cre recombinase and the PvuII endonuclease, the obtained strains MM219Cre pPvuII-1, MM219Cre pPvuII-3, MM219Cre pPvuII-6 were transformed with the compatible bireplicon plasmid pMlox3 using selection by kanamycin (Km). Eighteen clones harbouring pMlox3 and each of the plasmids pPvuII-1, pPvuII-3 and pPvuII-6 were grown in 5 ml of LB broth supplemented with Cm, Km and glucose. 2 ml of each culture were used for isolation of supercoiled DNA by the alkaline lysis protocol and of total DNA by the neutral lysis protocol. The remainders of the bacterial cultures were washed twice with a 1× solution of M9 salts, resuspended in 3 ml of a 1× solution of M9 salts supplemented with thiamin (1×g/ml), MgSO₄(1 mM), L-arabinose (0.5%) and incubated for additional 12 hours. The resultant cultures were used for isolation of supercoiled DNA by the alkaline lysis protocol and of total DNA by the neutral lysis protocol.

Agarose gel analyses of the isolated DNA indicated that DNA degradation after incubation with arabinose occurred in some of the clones harbouring pPvuII-6 but not in the clones harbouring pPvuII-1 or pPvuII-3. This result implies that the plasmid pPvuII-6 is able to provide for expression of PvuII in the presence of L-arabinose. No selective accumulation of minicircle DNA was expected in this experiment because all the pMlox3 recombination products are sensitive to PvuII.

In summary, the above experiments demonstrate that the pBAD promoter is not tight enough to control expression PvuII endonuclease in E. coli cells without appropriate methylation protection of the host DNA. 11 plasmids were selected containing intact PvuII gene and different rearrangements in pBAD promoter region. One of these mutant plasmids (pPvuII-6) can provide for tight control and substantial expression of PvuII after induction with L-arabinose.

Construction of Plasmid pMLOX6-Sp to Test for Minicircle DNA Accumulation

To test for preferential accumulation of minicircle DNA in a strain harbouring pPvuII-6, it was necessary to construct a minicircle producer plasmid with a minicircle module devoid of PvuII sites. To allow construction of the strain harbouring both pPvuII-6 and the new minicircle producer plasmid, the new plasmid should also have an antibiotic selection marker different from the marker of pPvuII-6 (Cm). It was also important to create a loxP66-polylinker-loxP71 module that is easily transferable to alternative vector backbones while retaining a variety of the cloning sites in the polylinker.

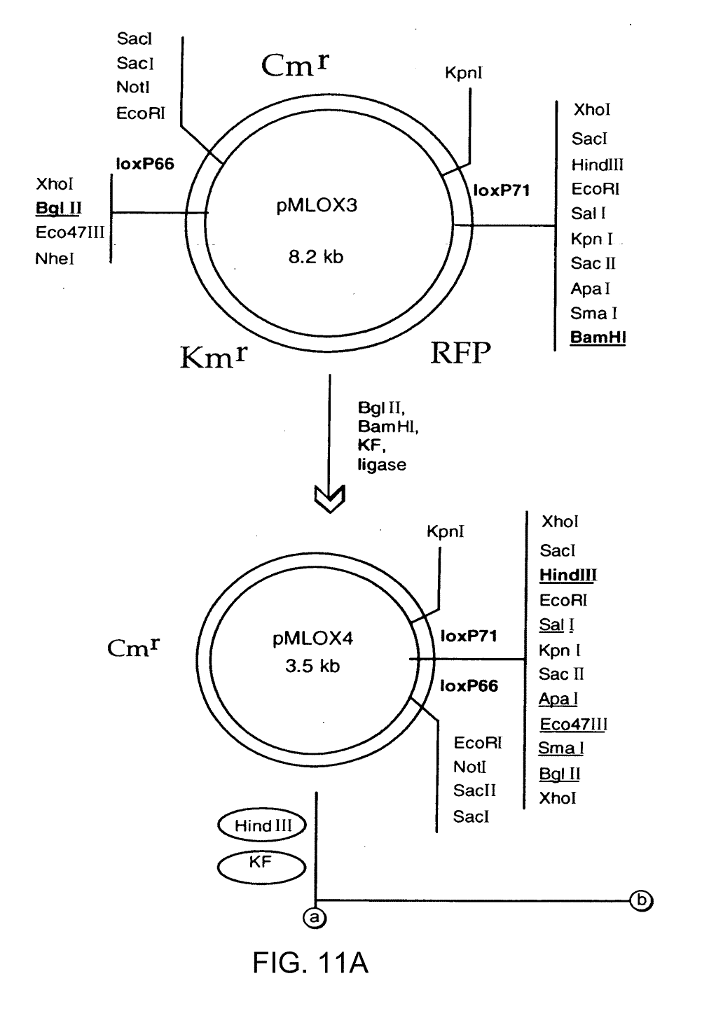

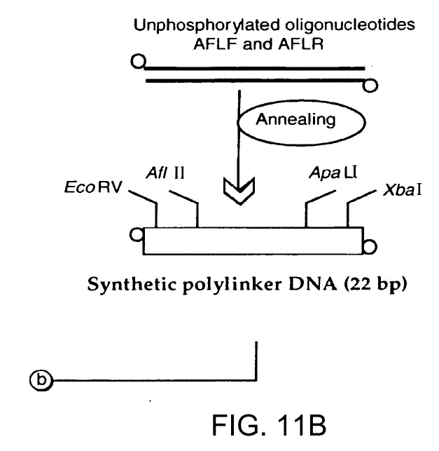

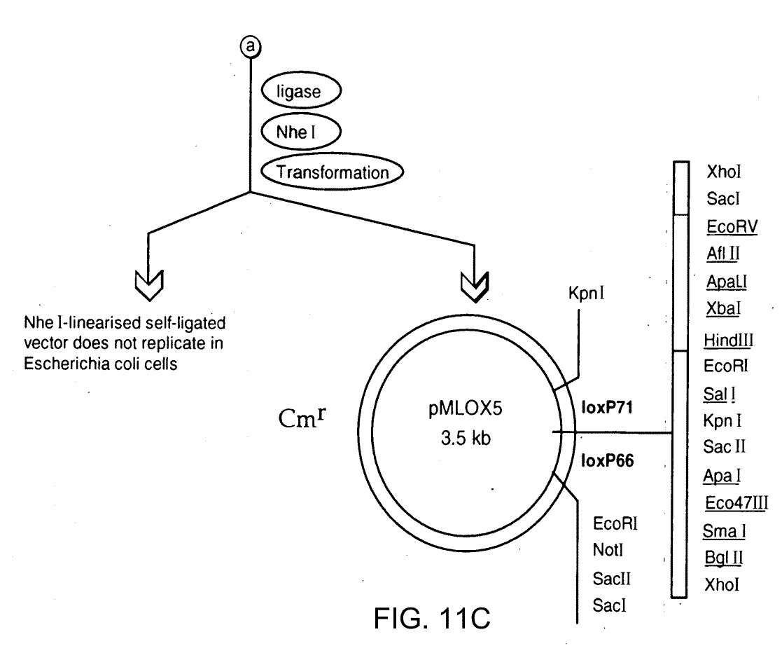

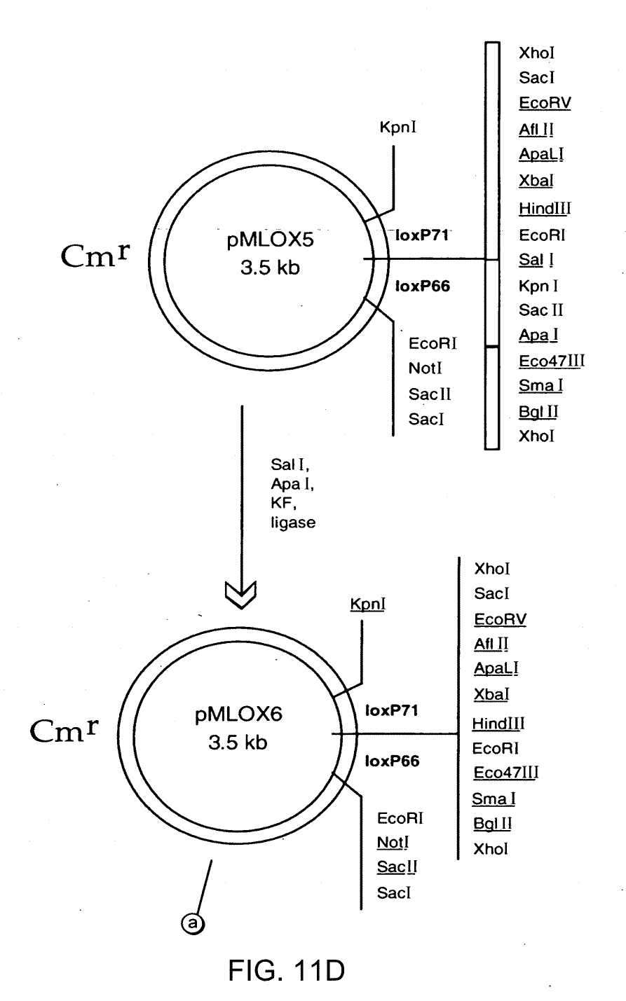

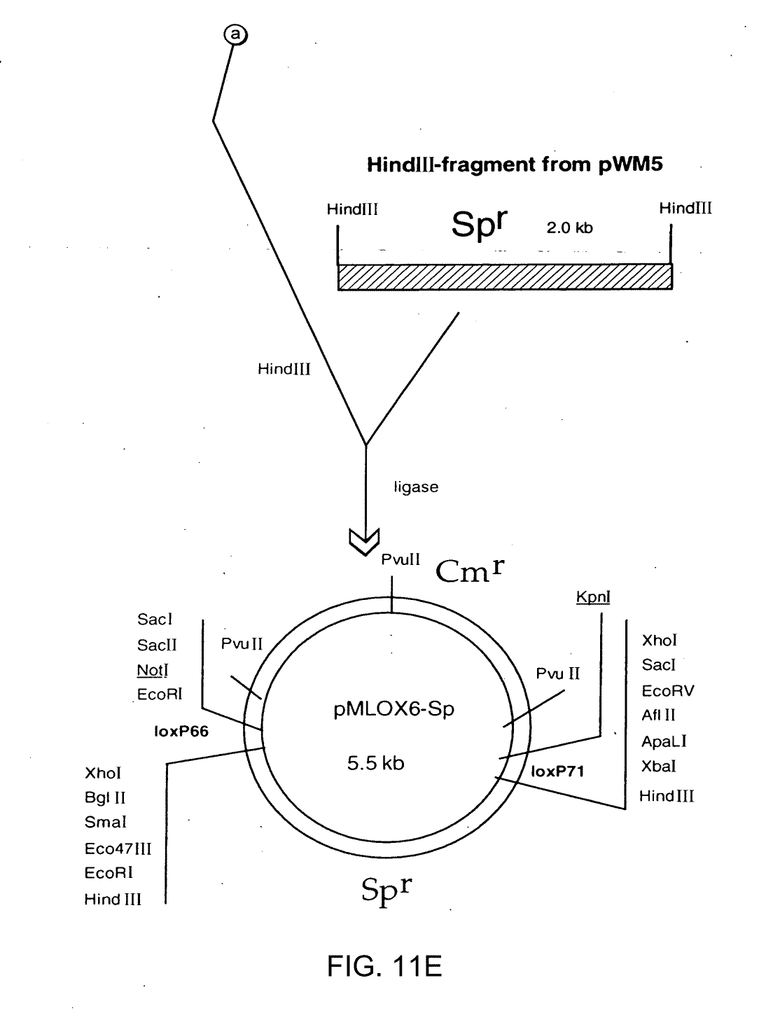

To address these requirements the Km/RFP moiety of pMlox3 was excised using BglII and BamHI to create pMLOX4. Then, the polylinker sequence in pMLOX4 was expanded, the unwanted KpnI site in the polylinker was removed and finally a HindIII-fragment with the spectinomycin (Sp) resistance gene from pWM5 (Metcalf & Wanner, 1993, Gene, 129(1): 17-25) was inserted into the unique HindIII site of the polylinker sequence (FIG. 11A to FIG. 11E). The resultant minicircle producer plasmid pMLOX6-Sp confers resistance to Sp and has a minicircle module that is devoid of PvuII sites. The minicircle module can be easily excised by NotI and KpnI for transfer onto other vector backbones with subsequent replacement of the Sp-resistance gene by a therapeutic or another gene of interest.

Preferential Accumulation of Minicircle DNA in the Strain MM219Cre Harbouring Plasmids pPvuII-6 and pMLOX6-Sp

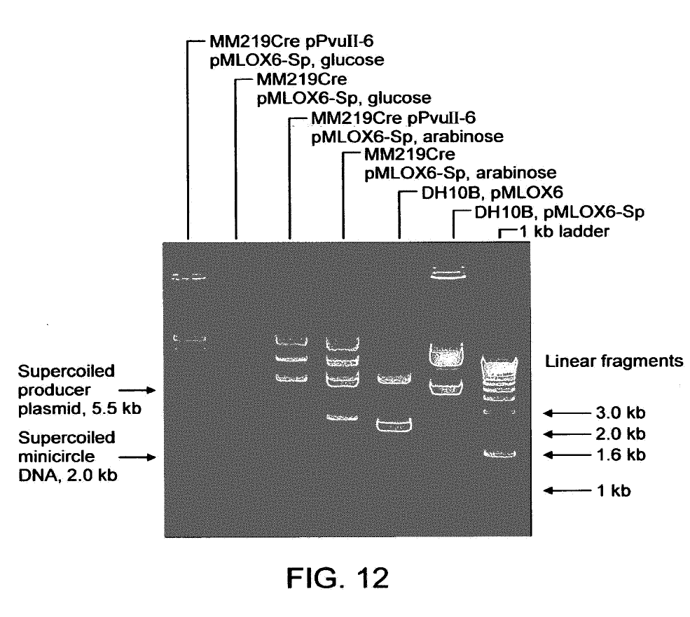

The obtained minicircle producer plasmid pMLOX6-Sp was introduced into MM219Cre strain harbouring pPvuII-6 by electroporation. The resultant transformants were checked for their inability to grow on LB agar supplemented with Cm (30 μg/ml), Sp (50 μg/ml) and L-arabinose (0.5%). In addition the plasmid pMLOX6-Sp was introduced into MM219Cre to generate a strain for control experiments. One of the MM219Cre pPvuII-6 pMLOX6-Sp clones and one of the MM219Cre pMLOX6-Sp clones were grown in 500 ml of LB broth supplemented with Cm, Sp and glucose. One and a half ml of each culture were used for isolation of supercoiled DNA by the alkaline lysis protocol. The remainder of the bacterial cultures was washed twice with a 1× solution of M9 salts, resuspended in 500 ml of a 1× solution of M9 salts supplemented with thiamin (1 μg/ml), MgSO₄ (1 mM), L-arabinose (0.5%) and incubated for additional 12 hours. The resultant cultures were used for isolation of supercoiled DNA by the alkaline lysis protocol. Agarose gel analysis indicated considerable degradation of the unresolved minicircle producer plasmid and the miniplasmid recombination product in the strain harbouring pPvuII-6 after incubation with arabinose (FIG. 12). Thus, we observed an overall selective accumulation of minicircle relative to the sum of all other products of Cre-recombination.

In summary, we have shown co-expression of an enzymatically active site-specific recombinase (Cre recombinase) and of a restriction enzyme (PvuII) in the minicircle DNA producing bacteria and the ensuing production and enrichment of minicircle DNA by their combined action. The incomplete destruction of the unwanted recombination products is probably due to the attenuated activity of PvuII provided by pPvuII-6 plasmid. An increased activity of the PvuII can be provided by a new plasmid containing the PvuII gene in anti-sense orientation where the induced site-specific recombinase activity is used to change anti-sense orientation of the PvuII gene to a sense orientation. Combination of catabolite repression of the site-specific recombinase under pBAD promoter and anti-sense orientation of PvuII gene will put spontaneous PvuII expression “under double lock” and thus ensure structural stability of the PvuII-plasmid and unattenuated expression of the PvuII endonuclase in the induced state.

### Example 3

Construction of Minicircle Producing Plasmids with attP and attB Sites for Bacteriophage ΦC31 Integrase

Integrase of bacteriophage ΦC31 is known to catalyse recombination between specific sequences called attP (39 bp) and attB (34 bp) (Thorpe & Smith, Proc Natl Acad Sci USA. 1998. 95(10): 5505-10; Groth et al, Proc Natl Acad Sci USA. 2000. 97(11): 5995-6000). The recombination sites attP and attB were introduced into plasmid pBC-SK(+) by annealing of oligonucleotides OLIGO-F 5′-CTCGAATTCA TAACTTCGTA TAGCATACAT TATACGAACG GTACTCGAGT ACCGTTCGTA TAGCATACAT TATACGAAGT TATGGTACCA AAAA-3′ and OLIGO-R 5′-TGATGAATTC CGCGCCCG-3′, filling in with Pfx-polymerase at 68° C., digesting further with EcoRI and KpnI, and ligating to create pDATT1. The unwanted polylinker was removed from pDATT1 by PstI and BamHI digestion, treatment with the Klenow fragment of the E. coli DNA polymerase I and ligation to produce pDATT2. The luciferase expression cassette was excised by XhoI from pFIXluc and inserted into the XhoI site between the attP and attB sites of pDATT2 to generate minicircle producer plasmid pDATT2luc. An indicator-plasmid to study integrase-mediated intramolecular recombination (pDATT2-Km) was created by insertion of the entire pDSRed1-N1 (Clontech) plasmid at XhoI between attP and attB of pDATT2.

Integrase of Bacteriophage ΦC31 Catalyses Intramolecular Recombination within Covalently Closed Circular and Linear DNA thus Allowing Production of Minicircle DNA.

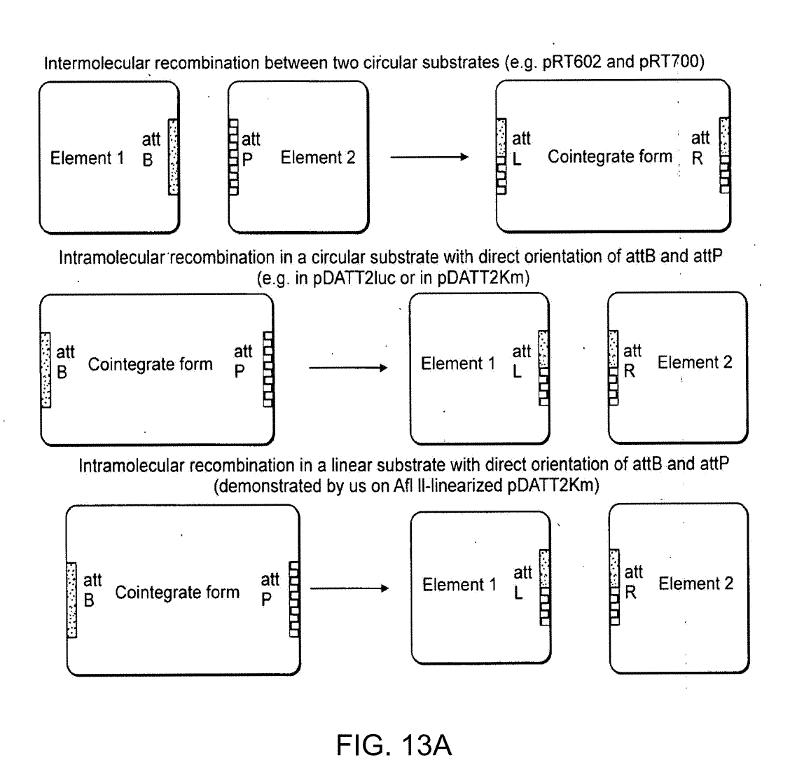

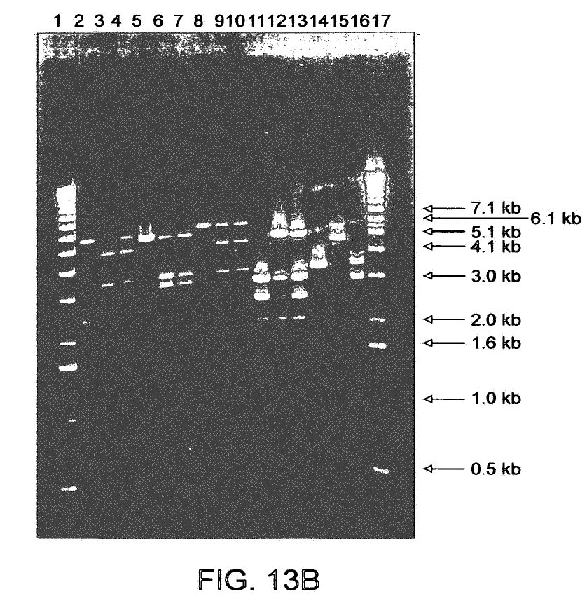

It was important to establish whether the ΦC31 integrase can catalyse intramolecular recombination on circular and linear DNA substrates (FIG. 13A). Uncut plasmids pDATT2luc, pDATT2-Km and AflII-linearised plasmid pDATT2-Km were treated with integrase of bacteriophage ΦC31 in vitro. The enzyme was produced by Dr. Maggie Smith (Nottingham University) from E. coli strain harbouring pHS62 plasmid. As a positive control, a mixture of uncut attB plasmid pRT602 and uncut attP plasmid pRT700 (Thorpe et al, Mol Microbiol. 2000. 38(2): 232-41) was treated with the integrase to show intermolecular recombination. The recombination products were digested with appropriate restriction enzymes to allow separation of catenated DNA molecules and were analyzed by electrophoresis in 0.7% agarose gel (FIG. 13B). The results show that the constructed plasmids pDATT2luc and pDATT2-Km were active in integrase-mediated recombination. Notably, the integrase was able to promote intramolecular site-specific recombination on linear substrate (AflII-linearised pDATT2-Km). Thus, hydrolysis of minicircle producer plasmids by PvuII or another endonuclease in vivo will not interfere with the integrase-mediated recombination reaction.

This experiment demonstrates that the integrase of bacteriophage ΦC31 can be used for minicircle DNA production. The intermolecular site-specific recombination catalysed by ΦC31 integrase was shown to occur in E. coli in vivo (Thorpe & Smith, Proc Natl Acad Sci USA. 1998. 95(10): 5505-10). We thus presume that intramolecular recombination and ensuing formation of minicircle DNA will be catalysed by ΦC31 integrase in E. coli cells in vivo. Thus, the gene for integrase can be excised from the above-mentioned plasmid pHS62 and inserted into pBAD75Cre to replace the Cre gene. The resultant plasmid pBAD75Int2 can be used for construction of the E. coli strains for production of minicircle DNA.

1. A method of producing a minicircle nucleic acid construct comprising:
(a) introducing a parental plasmid construct into a cell engineered to include the following components:
a genomically or episomally integrated coding sequence for a site-specific recombination enzyme,
a genomically or episomally integrated coding sequence for an endonuclease and/or exonuclease not endogenous to the cell operably linked to an endonuclease promoter,
wherein the cell is further engineered to express the coding sequence for the site-specific recombination enzyme and the coding sequence for the endonuclease and/or exonuclease not endogenous to the cell, and
wherein the parental plasmid construct comprises:
(i) an expression cassette including a gene sequence of interest flanked by two recombination sites specific to the recombination enzyme, and
(ii) at least one endonuclease and/or exonuclease site that is recognized by an endonuclease and/or exonuclease not endogenous to the cell located outside of the recombination sites and the expression cassette,
(b) exposing the cell to conditions for a period of time sufficient for the recombination enzyme to be expressed and recombine each respective recombination site; and sufficient for the endonuclease and/or exonuclease coding sequence to be expressed and digest the at least one endonuclease and/or exonuclease site not endogenous to the cell on the parental plasmid construct, wherein the exposing provides:
(i) a minicircle nucleic acid vector devoid of bacterial sequences and containing an expression cassette comprising the gene sequence of interest, and a first product hybrid recombination site sequence, and
(ii) a miniplasmid containing the bacterial sequences, the at least one endonuclease and/or exonuclease site that is recognized by the endonuclease and/or exonuclease not endogenous to the cell, and a second product hybrid recombination site sequence.

2. The method according to claim 1, wherein the recombination enzyme in the cell is operably linked to a recombination enzyme promoter.

3. The method according to claim 2, wherein each respective promoter operably linked to the coding sequence for the site-specific recombination enzyme and/or for the endonuclease and/or exonuclease not endogenous to the cell is a constitutive or inducible promoter.

4. The method according to claim 2, wherein the recombination enzyme promoter is selected from an arabinose expression system, an operator-repressor system of phage λ, an operator-repressor system of lac operon, or a tetracycline repressor-operator system.

5. The method according to claim 1, wherein the endonuclease promoter is selected from an arabinose expression system, an operator-repressor system of phage λ, an operator-repressor system of lac operon, a tetracycline repressor-operator system or a FLP site specific recombination system.

6. The method according to claim 1, wherein the recombination enzyme in the cell is operably linked to a promoter inducible with L-arabinose.

7. The method according to claim 1, wherein the endonuclease and/or exonuclease not endogenous to the cell is under the control of a promoter inducible with L-arabinose.

8. The method according to claim 1, wherein the recombination enzyme in the cell is an integrase, recombinase, yeast FLP, resolves or invertase.

9. The method according to claim 1, wherein the cell is a bacterium, mammalian or yeast cell.

10. The method according to claim 1, wherein the recombination enzyme is ΦC31 integrase and the recombination sites are attP and attB sites.

11. The method according to claim 1, wherein the recombination enzyme is Cre recombinase and both recombination sites are wild type loxP recombination sites.

12. The method according to claim 11, wherein one loxP recombination site sequence is modified at the 5′ end and the other loxP recombination site sequence is modified at the 3′ end.

13. The method according to claim 12, wherein the 5′ modified recombination site sequence is lox71 and the 3′ modified recombination site sequence is lox66.

14. The method according to claim 13, wherein after recombination, both of the respective modified loxP recombination site sequences form a lox71/lox66 product hybrid recombination site sequence in the minicircle nucleic acid construct.

15. The method according to claim 13, wherein after recombination, both of the respective unmodified loxP recombination site sequences form a wild type loxP product hybrid recombination sequence in the miniplasmid.

16. The method according to claim 1, wherein the parental plasmid construct is linear or circular.

17. The method according to claim 1, wherein the endonuclease is a DNA nuclease, preferably a restriction endonuclease or an intron-encoded endonuclease.

18. The method according to claim 1, wherein the miniplasmid and unrecombined parental plasmid are digested by the endonuclease and/or exonuclease not endogenous to the cell.

19. The method according to claim 1, wherein the minicircle nucleic acid construct contains at least one sequence of a mitochondrial, bacterial, viral, or mammalian sequence.

20. The method according to claim 1, wherein the expression of the gene sequence of interest occurs in a nucleus or in mitochondria.
